(19) United States
(12) Patent Application Publication
(10) Pub. No.: US 2024/0368404 A1
(43) Pub. Date: Nov. 7, 2024

(54) HYDROPHOBIC AND HYDROPHILIC MODIFIED MALEATED NATURAL OILS AND COMPOSITIONS THEREOF

(71) Applicant: ISP Investments LLC, Wilmington, DE (US)

(72) Inventors: OSAMA M. MUSA, Bedminster, NJ (US); Mousumi GHOSH, Elmwood Park, NJ (US); Donald I. PREttypaul, Englewood, NJ (US); Diane M. KENNEDY, Bayonne, NJ (US)

(73) Assignee: ISP Investments LLC, Wilmington, DE (US)

(21) Appl. No.: 18/689,718

(22) PCT Filed: Aug. 24, 2022
(86) PCT No.: PCT/US22/41337

§ 371 Date: Mar. 6, 2024

(51) Int. Cl. C08L 91/00; A61K 8/37; A61K 8/92; A61Q 19/00

(52) U.S. Cl. CPC C08L 91/00; A61K 8/375; A61K 8/922; A61Q 19/00

(57) ABSTRACT

The present invention provides compositions comprising a reaction product of (a) a maleated natural oil, comprising a natural oil with maleated functionality; and (b) a functionalized or unfunctionalized moiety selected from the group costing of hydrophobic moieties, hydrophilic moieties, and combinations thereof; with the proviso that the hydrophilic moiety is not a glycerol moiety

In another aspect of the invention, the invention provides compositions comprising a reaction product of (a) a maleated natural oil, comprising a natural oil with maleated functionality; (b) a functionalized or unfunctionalized hydrophobic moiety; and (c) a glycerol moiety.

In yet another aspect, the present application provides compositions comprising a reaction product of (a) a maleated natural oil, comprising a natural oil with maleated functionality; (b) two functionalized or unfunctionalized hydrophilic moieties.

The above compositions may further comprise a functional system active ingredient. The modified maleated natural oils are useful in a wide variety of compositions and applications.

In yet another aspect of the invention, the invention provides the compound represented by the structures set out below:

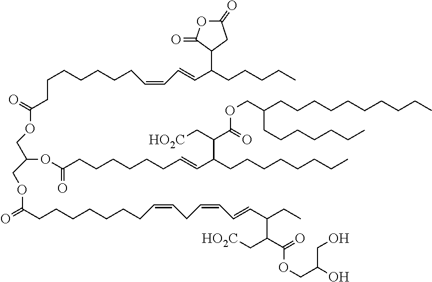 [SMILES: CCCCCCCCCCC(CCCCCCC)COC(=O)C(CC(=O)O)C(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C(CC(=O)O)C(=O)OCC(O)CO)CCCCCCCC]

### FIELD OF THE INVENTION

The present application provides compositions comprising modified maleated natural oils. The modified maleated natural oils do not exhibit many of the limited properties of maleated natural oils. The modified maleated natural oils are useful in a wide variety of compositions and applications.

### BACKGROUND OF THE INVENTION

Modified natural oils can be non-dispersible in water or alcohols. As a result, these oils can be incorporated into a wide variety of compositions. Such compositions include, but are not limited to, personal care (e.g., hair care, sun care, skin care, oral care), adhesives, coatings, paints, electronics, household, industrial and institutional (HI&I) compositions, inks, membranes, metal working fluids, oilfield chemicals, plastics and plasticizers, textiles, industrial products, biocides, pharmaceuticals/nutritional s, and agrochemical compositions.

Natural oils, such as soybean and linseed oils, are one of the most promising raw materials for the synthesis of renewable compounds, including polymers, plastics, and plasticizers. These natural materials are inexpensive, highly abundant, come from reliable and sustainable sources, and have high potential for modification. Natural oils are generally blends of different triglycerides, the esterification product of fatty acids and glycerol, and contain varying degrees of unsaturation (i.e., double bonds). Oils can be characterized by a hydroxyl value and the fatty acid compositions. Both natural fatty acids and natural oils must be chemically modified to make them sufficiently reactive to allow structural alterations and polymerizations to occur because the olefin functional groups are relatively unreactive. Unsaturated double bonds in these compounds have been converted to epoxide functional groups and succinic anhydride functional groups, allowing the addition of many hydroxyl containing species to be introduced into the natural oils.

Maleated natural oils are natural oils that have been chemically functionalized by the chemical addition of epoxide (oxirane) and succinic anhydride functional groups. Examples include epoxidized and maleated soybean oil and linseed oil, unsaturated natural oils lend themselves to these chemical functionalizations.

U.S. Pat. No. 9,809,538B2 describes a modified natural compound synthesized from epoxidized natural fatty acid, maleated natural fatty acid, epoxidized natural oil, or maleated natural oil; and lactam compound with hydroxyl(s) to form e.g. adhesive or beverage composition.

A discussion of reaction scheme for the maleinization reaction in the vegetable oil is provided in the article “Maleated soybean oil and its multifunctional properties,” by Gripp, Anna A., Steinberg, David C., published in Cosmetics Exhibition & Conference Conference Proceedings, Barcelona, Mar. 22-24, 1994.

A discussion of reaction scheme for the maleinization reaction in the vegetable oil is provided in the article “Microwave Assisted Syntheses of Vegetable Oil Based Monomer,” by Rafael T. Alarcon et al., published in Journal of Polymers and the Environment 28:1265-1278, 2020.

A discussion of generalized reaction between maleic anhydride and unsaturated vegetable oils is provided in the handbook of maleic Anhydride based materials-syntheses, properties and applications by Osama M. Musa in chapter 3 page 166, published in Springer International Publishing Switzerland 2016.

U.S. Pat. No. 2,754,306A describes a reaction with a soybean oil, maleic anhydride based and iso-octyl alcohol to provide an improved plasticizer for nitrocellulose compositions.

PCT Application 2019113068A1 and 2005071050A1 describes a technology related to metalworking fluids comprising maleated soybean oil derivatives.

A discussion of maleic anhydride polymerization and modified plant oils with polyols is provided in the article “Polymerization of Maleic Anhydride-Modified Plant Oils with Polyols,” by Tarik Eren, Selim H. Kusefoglu, Richard Wool published in Journal of Applied Polymer Science, Barcelona, Volume 90, Issue 1, Pages 197-202, 2003.

EP Patent 2754306A describes an adhesive containing a polycondensate and a dienophile modified fatty acid as a cross-linking agent.

US Publication 20180070584A1 describes an adjuvant composition that includes a maleated natural oil derivative into agrochemical formulations and applied to target substrates to kill, inhibit, or repel pests.

PCT Application 2005071050A1 describes a metalworking fluid comprising oil in water emulsion from a reaction product of maleic anhydride and a triglyceride oil from a plant or land animal and further reacted with water, Group IA and IIA metals, ammonium hydroxide, various amines, alkanolamines, polyols, alkoxylated alkanolamines, poly(alkylene oxide)s, or polyamines or mixtures.

Despite the renewability, biodegradability, sustainability, and beneficial functions provided by natural fatty acids, natural oils and their maleated counterparts, they exhibit properties that can limit their application. For example, maleated soybean oil is insoluble and non-dispersible in water or alcohols. As a result, these oils may tend to exude or phase-separate from formulated compositions. This feature makes their formulation more difficult, often requiring additional ingredients to facilitate solutions, emulsions, or dispersions. Natural and maleated natural oils may not impart the desired property needed in end-uses, such as solubilization capability, glass transition, flexibility, shine, and/or plasticization. Consequently, the performance (including, but not limited to stability, resistance to phase separation, absorption, clean-up, solubility potential, staining potential, lubrication, film formation, uniformity of spreading, comedogenic tendency, ease of removal), and aesthetic qualities (such as skin-feel, greasiness, shiny appearance) may be less than desired. Finally, although such natural oils are an important renewable material, they are not always the formulator's first choice, and, in fact, often are not considered at all.

Mineral oils and vegetable oils are often used as components of personal care compositions. Accordingly, there is a need for materials that are renewable, natural, and biodegradable having different and controllable chemical, physical, and/or mechanical properties such that the limitations found in natural and maleated natural oils are minimized or eliminated. Most personal care compositions contain more than one inert ingredient to help deliver the active ingredient to the desired skin care properties. Others are persistent in the skin care having toxic products or other undesired side-effects. In addition, each time a new active component is released to the market, it necessitates the development of a unique composition to get the active ingredient into the field. Therefore, there is a need to develop new, functionalized maleated natural oils for use in personal care inert ingredients that are non-toxic and inspired by or created from renewable resources.

### SUMMARY OF THE INVENTION

The present invention provides compositions comprising a reaction product of (a) a maleated natural oil, comprising a natural oil with maleated functionality; and (b) a functionalized or unfunctionalized moiety selected from the group costing of hydrophobic moieties, hydrophilic moieties, and combinations thereof, with the proviso that the hydrophilic moiety is not a glycerol moiety

In another aspect of the invention, the invention provides compositions comprising a reaction product of (a) a maleated natural oil, comprising a natural oil with maleated functionality; (b) a functionalized or unfunctionalized hydrophobic moiety; and (c) a glycerol moiety.

In yet another aspect, the present application provides compositions comprising (A) a reaction product of: (a) a maleated natural oil, comprising a natural oil with maleated functionality; and (b) a functionalized or unfunctionalized moiety selected from the group consisting of hydrophobic moieties, hydrophilic moieties, and combinations thereof, and (B) (a) a functional system active ingredient.

In yet another aspect, the present application provides compositions comprising a reaction product of
(a) a maleated natural oil, comprising a natural oil with maleated functionality;
(b) two functionalized or unfunctionalized hydrophilic moieties.

In yet another aspect, the present application provides compositions comprising:
(A) a reaction product of
(a) a maleated natural oil, comprising a natural oil with maleated functionality;
(b) two functionalized or unfunctionalized hydrophilic moieties; and
(B) a functional system active ingredient.

In yet another aspect of the invention, the invention provides compounds represented by the structures set out below:

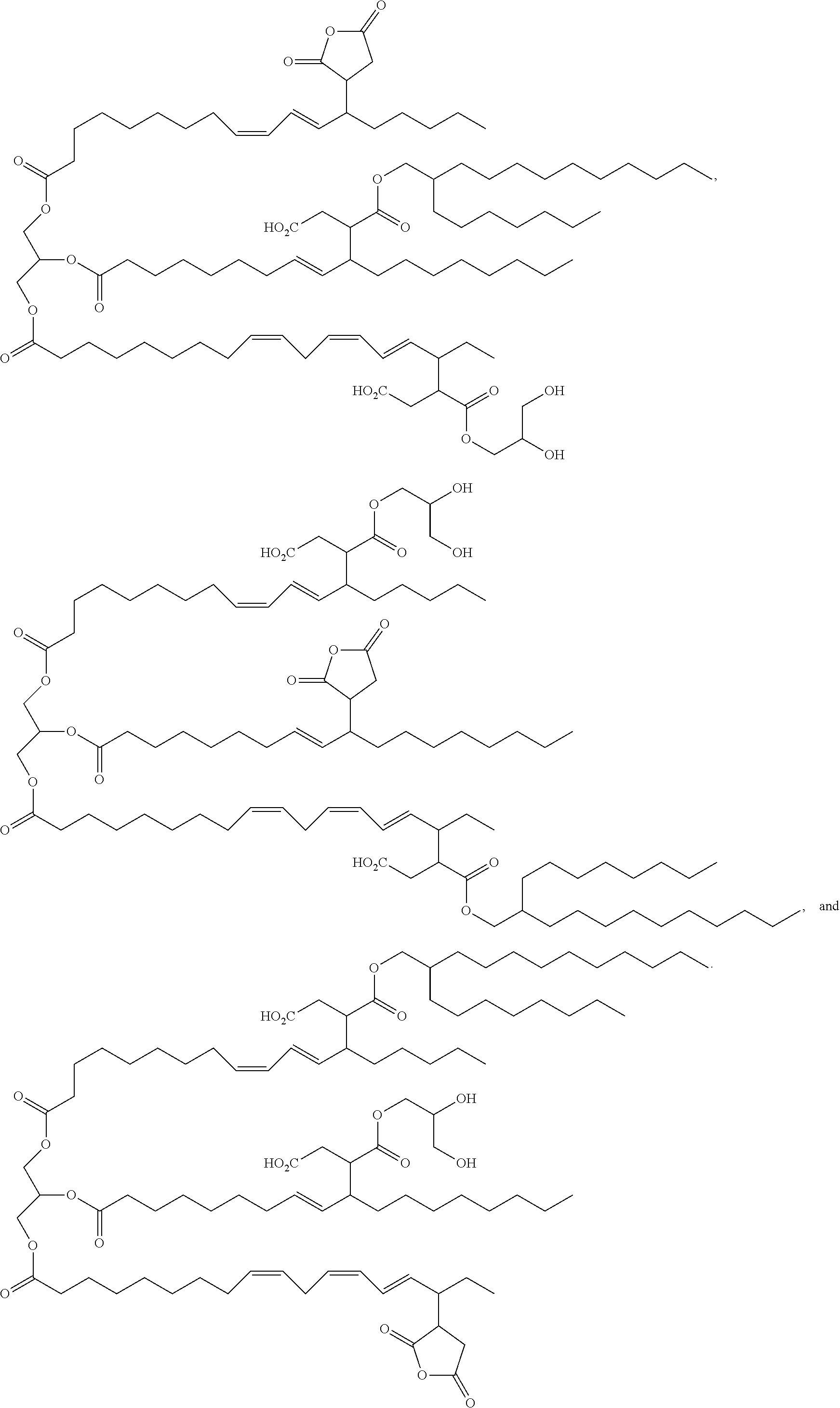

The modified maleated natural oils are useful in a wide variety of compositions and applications.

### DETAILED DESCRIPTION OF THE INVENTION

The present application provides compositions comprising modified maleated natural oils which do not exhibit many of the limited properties of maleated natural oils. The modified maleated natural oils are useful in a wide variety of compositions and applications.

Natural oils are abundant, inexpensive, and are derived from sustainable sources. Natural oils are useful for synthesizing renewable compounds such as polymers, plastics, and plasticizers, which compounds are useful in a variety of compositions. A difficultly with utilizing natural oils is that they are blends of triglycerides containing varying degrees of unsaturated groups, which unsaturated groups are relatively unreactive. To make these natural oils reactive, these unsaturated groups are generally chemically modified to make them reactive. For example, these unsaturated groups can be reacted with maleates to provide epoxide functional groups and succinic anhydride functional groups. Despite these chemical modifications, the maleated natural oils can still exhibit limited properties such as insolubility or non-dispersibility in water and alcohols. Accordingly, there is a need for further modified maleated natural oils which do not exhibit the limited properties of maleated natural oils.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

The term “branched and unbranched alkyl groups” refers to alkyl groups, which may be straight chained or branched. Branched groups include isopropyl, tert-butyl, and the like.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

The term “polymer” refers to a compound comprising repeating structural units (monomers) connected by covalent chemical bonds. Polymers may be further derivatized, crosslinked, grafted or end-capped. Non-limiting examples of polymers include copolymers, terpolymers, tetrapolymers, quaternary polymers, and homologues. The term “copolymer” refers to a polymer consisting essentially of two or more different types of monomers polymerized to obtain said copolymer.

The term “reaction product” refers to a substance produced from a chemical reaction of one or more reactant substances.

The term “natural oil” refers to compounds comprising triglycerides and may contain varying levels of fatty acids, monoglycerides, diglycerides and triglycerides refer to oil derived from plants or animal sources. Natural oils also include fatty acid glyceryl esters, which are synthesized by reacting glycerol with 1, 2, or 3 molar equivalents of a fatty acid or a mixture of fatty acids. These compounds can be mono, di or triglycerides of a single fatty acid or a mixture of fatty acids.

The term “maleated natural oil”, as used herein, refers to natural oil contains at least one or more maleated functionalities. Accordingly, the term “maleation” or “maleated” as used hereafter should be understood to mean “functionalization” insofar as the use of functionalizing reagents other than maleic anhydride are contemplated for use in the process of the invention.

As used herein, the term “moiety” or “moieties” refers to a part or a functional group(s) of a molecule.

The term “functional system active ingredient” refers to any ingredient that provides pharmacological activity or other direct effect in the diagnosis, cure, mitigation, treatment, or prevention of disease, or affects the structure or any function of the body of man or animals. The functional system active ingredient may be present either in a personal care product or a household care product. When the functional system active ingredient is present in a personal care product that contains at least one active personal care ingredient, the personal care active ingredient includes, but is not limited to, analgesics, anesthetics, antibiotic agents, antifungal agents, antiseptic agents, antidandruff agents, antibacterial agents, vitamins, hormones, antidiarrhea agents, corticosteroids, anti-inflammatory agents, vasodilators, kerolytic agents, dry-eye compositions, wound-healing agents, anti-infection agents, as well as solvents, diluents, adjuvants and other ingredients such as water, mineral oils, preservatives, surfactants, propellants, fragrances, essential oils, and viscosifying agents.

The term “functionalized” with reference to any moiety refers to the presence of one or more functional groups in the moiety. Various functional groups may be introduced in a moiety by way of one or more functionalization reactions known to a person having ordinary skill in the art. Non-limiting examples of functionalization reactions include: alkylation, epoxidation, sulfonation, hydrolysis, amidation, esterification, hydroxylation, dihydroxylation, amination, ammonolysis, acylation, nitration, oxidation, dehydration, elimination, hydration, dehydrogenation, hydrogenation, acetalization, halogenation, dehydrohalogenation, Michael addition, aldol condensation, Canizzaro reaction, Mannich reaction, Clasien condensation, Suzuki coupling, and the like. In one non-limiting embodiment, the term “functionalized” with reference to any moiety refers to the presence of one more functional groups selected from the group consisting of alkyl, alkenyl, hydroxyl, carboxyl, halogen, alkoxy, amino, imino, and combinations thereof, in the moiety.

As used herein, the term “hydrophilic” means that the compound has an affinity for water, whereas “hydrophobic” means not having an affinity for water.

The terms are relative terms, where a hydrophilic moiety it has a higher affinity for water than a hydrophobic moiety, but the hydrophilic moiety may or may not be completely water soluble. Likewise, hydrophobic moieties have less of an affinity for water than hydrophilic moieties, but the hydrophobic moieties may not necessarily be water-repellant. While hydrophilic moieties have an affinity for water and other polar solvents, hydrophobic moieties tend to have an affinity for oils, fats, and other non-polar solvents.

The term “unreacted maleated functionality” refers to a composition comprising a reaction product of maleated natural oil in which the components of maleated functionality are completely unreacted and properties are not changed. The term “hydrocarbyl” includes straight-chain and branched-chain alkyl, alkenyl, alkynyl, cycloalkyl, cycloalkenyl, aryl groups, and combinations thereof with optional heteroatom(s). A hydrocarbyl group may be mono-, di- or polyvalent and have carbon chains contain at least 2 carbon atoms and preferably 2 to 100 carbon atoms.

The term “alkyl” refers to a functionalized or unfunctionalized, monovalent, straight-chain, branched-chain, or cyclic C₁-C₆₀ hydrocarbyl group optionally having one or more heteroatoms. In one non-limiting embodiment, an alkyl is a C₁-C₄₅ hydrocarbyl group. In another non-limiting embodiment, an alkyl is a C₁-C₃₀ hydrocarbyl group. Non-limiting examples of alkyl include methyl, ethyl, n-propyl, isopropyl, n-butyl, isobutyl, tert-butyl, n-pentyl, isopentyl, n-hexyl, n-heptyl, n-octyl, 2-ethylhexyl, tert-octyl, iso-norbornyl, n-dodecyl, tert-dodecyl, n-tetradecyl, n-hexadecyl, n-octadecyl, n-eicosyl, cyclobutyl, cyclopentyl, cyclohexyl, and the like. The definition of “alkyl” also includes groups obtained by combinations of straight-chain, branched-chain and/or cyclic structures.

The term “aryl” refers to a functionalized or unfunctionalized, monovalent, aromatic hydrocarbyl group optionally having one or more heteroatoms. The definition of aryl includes carbocyclic and heterocyclic aromatic groups. Non-limiting examples of aryl groups include phenyl, naphthyl, indenyl, indanyl, azulenyl, fluorenyl, anthracenyl, furyl, thienyl, pyridyl, pyrrolyl, oxazolyl, thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, cinnolinyl, phthalazinyl, quinazolinyl, quinoxalinyl, 1,8-naphthridinyl, pteridinyl, carbazolyl, acridinyl, phenazinyl, phenothiazinyl, phenoxyazinyl, pyrazolo[1,5-c]triazinyl, and the like.

The term “aralkyl” refers to an alkyl group comprising one or more aryl substituent(s) wherein “aryl” and “alkyl” are as defined above. Non-limiting examples of aralkyl groups include benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like.

The term “alkylene” refers to a functionalized or unfunctionalized, divalent, straight-chain, branched-chain, or cyclic C₁-C₄₀ hydrocarbyl group optionally having one or more heteroatoms. In one non-limiting embodiment, an alkylene is a C₁-C₃₀ group. In another non-limiting embodiment, an alkylene is a C₁-C₂₀ group. Non-limiting examples of alkylene groups include:

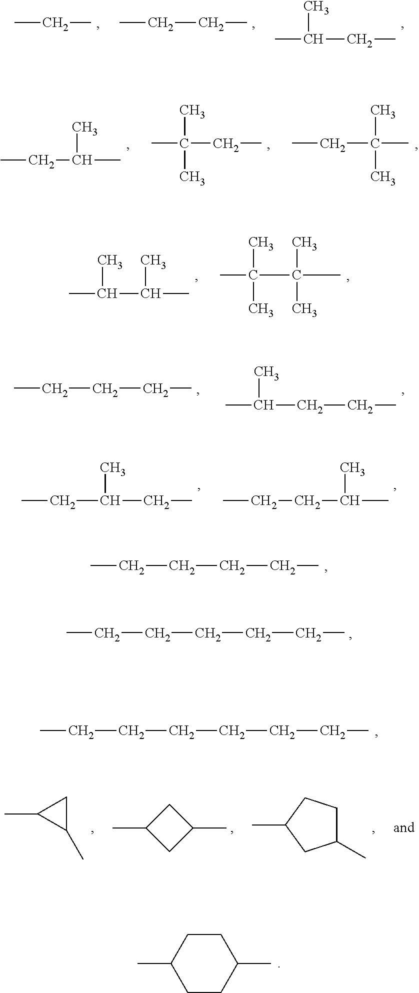

The term “arylene” refers to a functionalized or unfunctionalized, divalent, aromatic hydrocarbyl group optionally having one or more heteroatoms. The definition of arylene includes carbocyclic and heterocyclic groups. Non-limiting examples of arylene groups include phenylene, naphthylene, pyridinylene, and the like.

The term “heteroatom” refers to oxygen, nitrogen, sulfur, silicon, phosphorous, or halogen. The heteroatom(s) may be present as a part of one or more heteroatom-containing functional groups. Non-limiting examples of heteroatom-containing functional groups include ether, hydroxy, epoxy, carbonyl, carboxamide, carboxylic ester, carboxylic acid, imine, imide, amine, sulfonic, sulfonamide, phosphonic, and silane groups. The heteroatom(s) may also be present as a part of a ring such as in heteroaryl and heteroarylene groups.

The structures set out below may refer to the first, second, or third structure, or combinations thereof. The succinic anhydride group may be present in the top, middle, or lower chain.

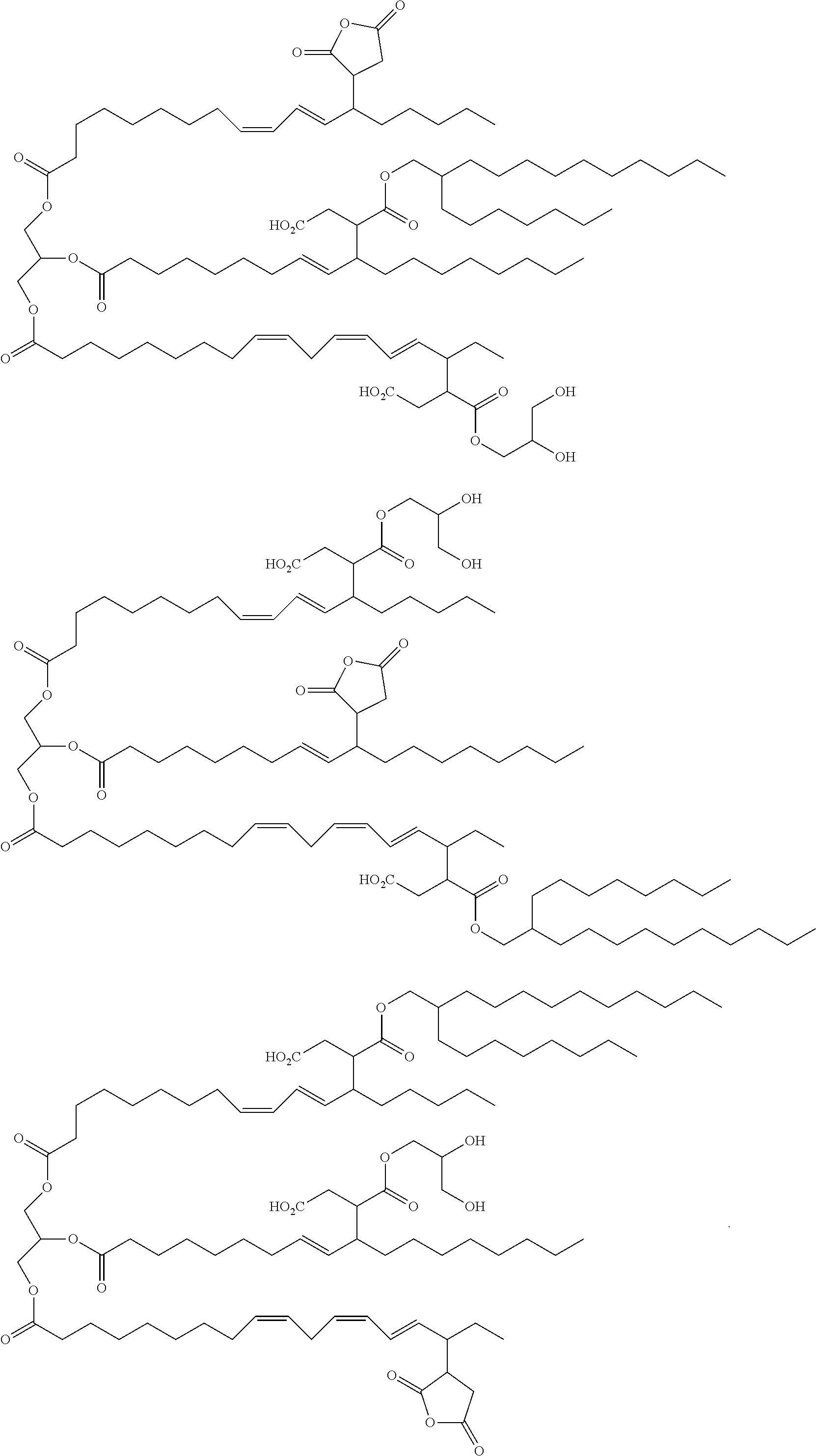

In one non-limiting embodiment, the hydrophobic moiety and the hydrophilic moiety is a hydrocarbyl alcohol, a hydrocarbyl amine, a silicon-based compound, or a combination thereof.

Hydrocarbyl alcohols are classified as primary, secondary and tertiary alcohols, based on the number of carbon atoms connected to the carbon atom that bears the hydroxyl group. Each classification of alcohol may have a general formula. For example,
the general formula for primary alcohols is

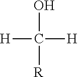

the general formula for secondary alcohols is

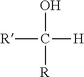

and
the general formula for tertiary alcohols is

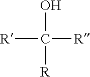

wherein R, R′ and R″ stand for hydrogen, different alkyl, alkylene, aryl, aralkyl, arylene, heteroatom groups.

The present invention provides compositions comprising a reaction product of (a) a maleated natural oil, comprising a natural oil with maleated functionality; and (b) a functionalized or unfunctionalized moiety selected from the group consisting of hydrophobic moieties, hydrophilic moieties, and combinations thereof, with the proviso that the hydrophilic moiety is not a glycerol moiety. Preferably, the reaction product comprises an unreacted maleated functionality or a maleated functionality functionalized with a hydrophobic moiety, a hydrophilic moiety, or combinations thereof.

Preferably, the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof.

Preferably, the hydrophilic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₁ to about C₅ atoms, unsubstituted or substituted alkyl, cycloalkyl, alkenyl, and aryl amines, wherein any of the beforehand mentioned groups may be with or without heteroatoms, containing from about C₁ to about C₅ atoms, unsubstituted or substituted polyols, wherein any of the beforehand mentioned groups may be with or without heteroatoms, containing from about C₂ to about C₃₆ atoms, silanes, and combinations thereof.

Preferably, the silane is functionalized with an alcohol or an amine, and combinations thereof. More preferably, the hydrophobic alcohol is selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof. More preferably, the hydrophilic alcohol is selected from the group consisting of methanol, ethanol, propanol, isopropanol, butanol, ethylene glycol, propylene glycol, diethylene glycol, dipropylene glycol, dibutylene glycol, polyethylene glycol, methoxypolyethylene glycol, polypropylene glycol, hexylene glycol, sorbitol, neopentylglycol, eythritol, mannitol, xylitol, threitol, pentaerythritol, beta-cyclodextrin, L-Ribose, 2-deoxy-D-galactose, and mixtures thereof.

Preferably, the hydrophobic amine is selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof. Preferably, the hydrophilic amine is selected from the group consisting of 2-methylpentane-1,5-diamine, diethanol amine, diisopropanolamine, serinol hydrochloride, 2-amino-2-ethyl-1,3-propanediol, N-methyl-D-glucosamine, D-galactosamine hydrochloride, D-glucosamine hydrochloride, D-mannosamine hydrochloride, and mixtures thereof.

Preferably, the silicon-based compound is a hydrophobic compound selected from the group consisting of aminopropylmethylsiloxane-dimethylsiloxane, N-ethylaminoisobutyl terminated polydimethylsiloxane, poly(1,1-dimethyl silazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof. Preferably, the silane is a hydrophilic compound selected from the group consisting of 3-aminopropylsilanetriol, N-(2-aminoethyl)-3-aminopropylsilanetriol, and mixtures thereof.

Preferably, the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof. More preferably, the maleated natural oil is a maleated soybean oil.

Preferably, the composition is selected from the group consisting of skin care compositions, oral care compositions, hair care compositions, energy compositions, construction compositions, biocidal compositions, preservative compositions, nutraceutical compositions, food compositions, agricultural compositions, coating compositions, cosmetic compositions, homecare compositions, industrial and institutional compositions, textile compositions, laundry compositions, cleaning compositions, and disinfecting compositions.

In another aspect, the present invention provides a composition comprising a reaction product of (a) a maleated natural oil, comprising a natural oil with maleated functionality; (b) a functionalized or unfunctionalized hydrophobic moiety; and (c) a glycerol moiety. Preferably, the reaction product comprises an unreacted maleated functionality or a maleated functionality functionalized with a hydrophobic moiety, a hydrophilic moiety, or combinations thereof.

Preferably, the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof. Preferably, the hydrophobic moiety is an alcohol selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof. Preferably, the hydrophobic moiety is an amine selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof.

Preferably, the silicon-based compound is a hydrophobic compound selected from the group consisting of aminopropylmethylsiloxane-dimethylsiloxane, N-ethylaminoisobutyl terminated polydimethylsiloxane, poly(1,1-dimethyl silazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

Preferably, the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof. More preferably, the maleated natural oil is a maleated soybean oil.

Preferably, the composition is selected from the group consisting of skin care compositions, oral care compositions, hair care compositions, energy compositions, construction compositions, biocidal compositions, preservative compositions, nutraceutical compositions, food compositions, agricultural compositions, coating compositions, cosmetic compositions, homecare compositions, industrial and institutional compositions, textile compositions, laundry compositions, cleaning compositions, and disinfecting compositions.

A preferred composition comprises compounds selected from the group of structures represented by the structures set out below:

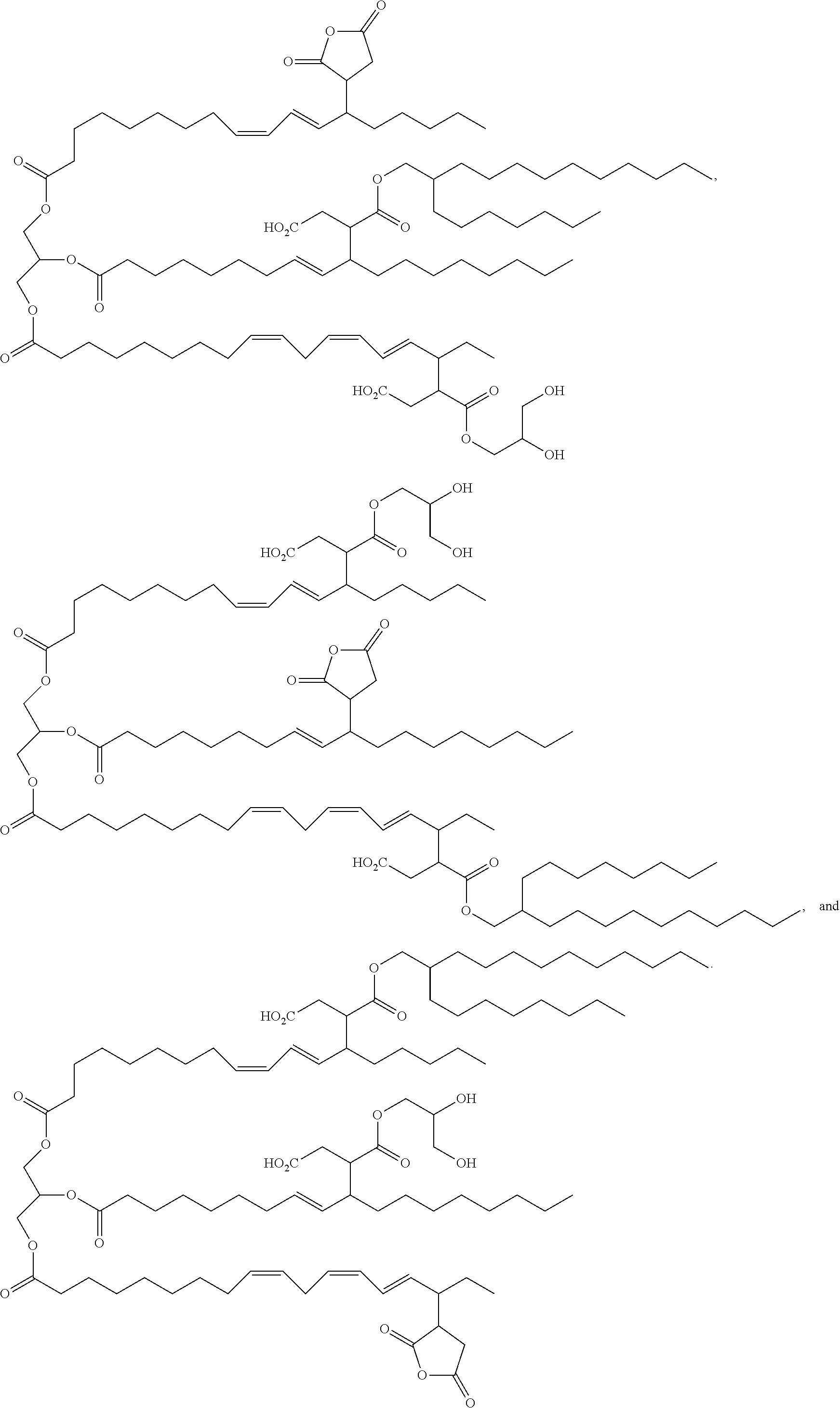

A preferred compound may be selected from the group of structures represented by the structures set out below:

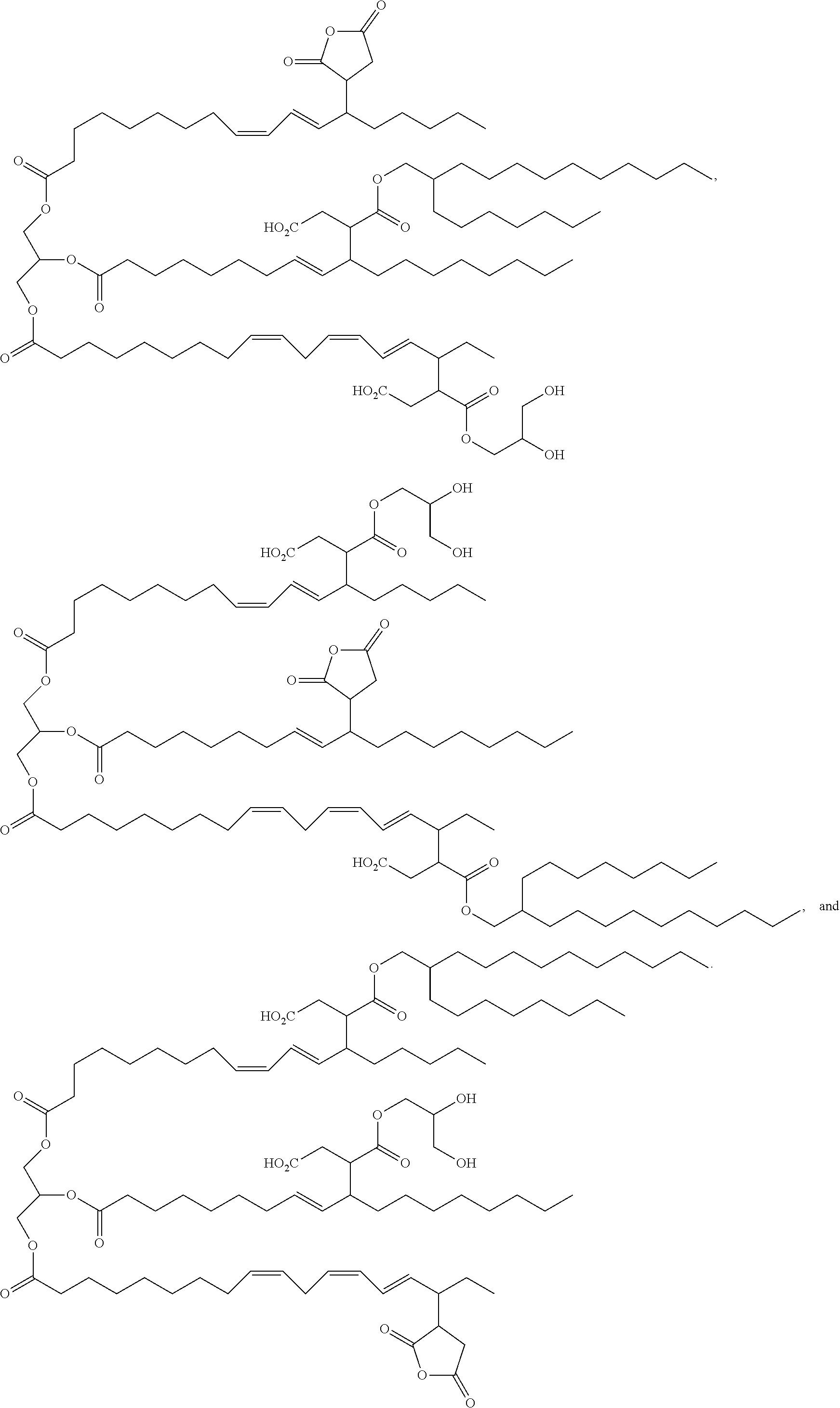

In yet another aspect, the invention provides a composition comprising: (A) a reaction product of: (a) a maleated natural oil, comprising a natural oil with maleated functionality; and (b) a functionalized or unfunctionalized moiety selected from the group consisting of hydrophobic moieties, hydrophilic moieties, and combinations thereof, with the proviso that the hydrophilic moiety is not a glycerol moiety, and (B) (a) a functional system active ingredient.

The preferences listed for the invention set out above for a “composition comprising a reaction product of (a) a maleated natural oil, comprising a natural oil with maleated functionality; and (b) a functionalized or unfunctionalized moiety selected from the group consisting of hydrophobic moieties, hydrophilic moieties, and combinations thereof, with the proviso that the hydrophilic moiety is not a glycerol moiety” also apply to this aspect of the invention.

The compositions may be selected from the group consisting of skin care compositions, oral care compositions, hair care compositions, energy compositions, construction compositions, biocidal compositions, preservative compositions, nutraceutical compositions, food compositions, agricultural compositions, coating compositions, cosmetic compositions, homecare compositions, industrial and institutional compositions, textile compositions, laundry compositions, cleaning compositions, and disinfecting compositions.

In yet another aspect, the invention provides a composition comprising: (A) a reaction product of (a) a maleated natural oil, comprising a natural oil with maleated functionality; (b) a functionalized or unfunctionalized hydrophobic moiety; (c) a glycerol moiety; and (B) a functional system active ingredient.

The preferences listed for the invention set out above for a “composition comprising: (A) a reaction product of (a) a maleated natural oil, comprising a natural oil with maleated functionality; (b) a functionalized or unfunctionalized hydrophobic moiety; (c) a glycerol moiety; and (B) a functional system active ingredient” also apply to this aspect of the invention.

The compositions may be selected from the group consisting of skin care compositions, oral care compositions, hair care compositions, energy compositions, construction compositions, biocidal compositions, preservative compositions, nutraceutical compositions, food compositions, agricultural compositions, coating compositions, cosmetic compositions, homecare compositions, industrial and institutional compositions, textile compositions, laundry compositions, cleaning compositions, and disinfecting compositions.

A preferred composition comprises compounds selected from the group of structures represented by the structures set out below:

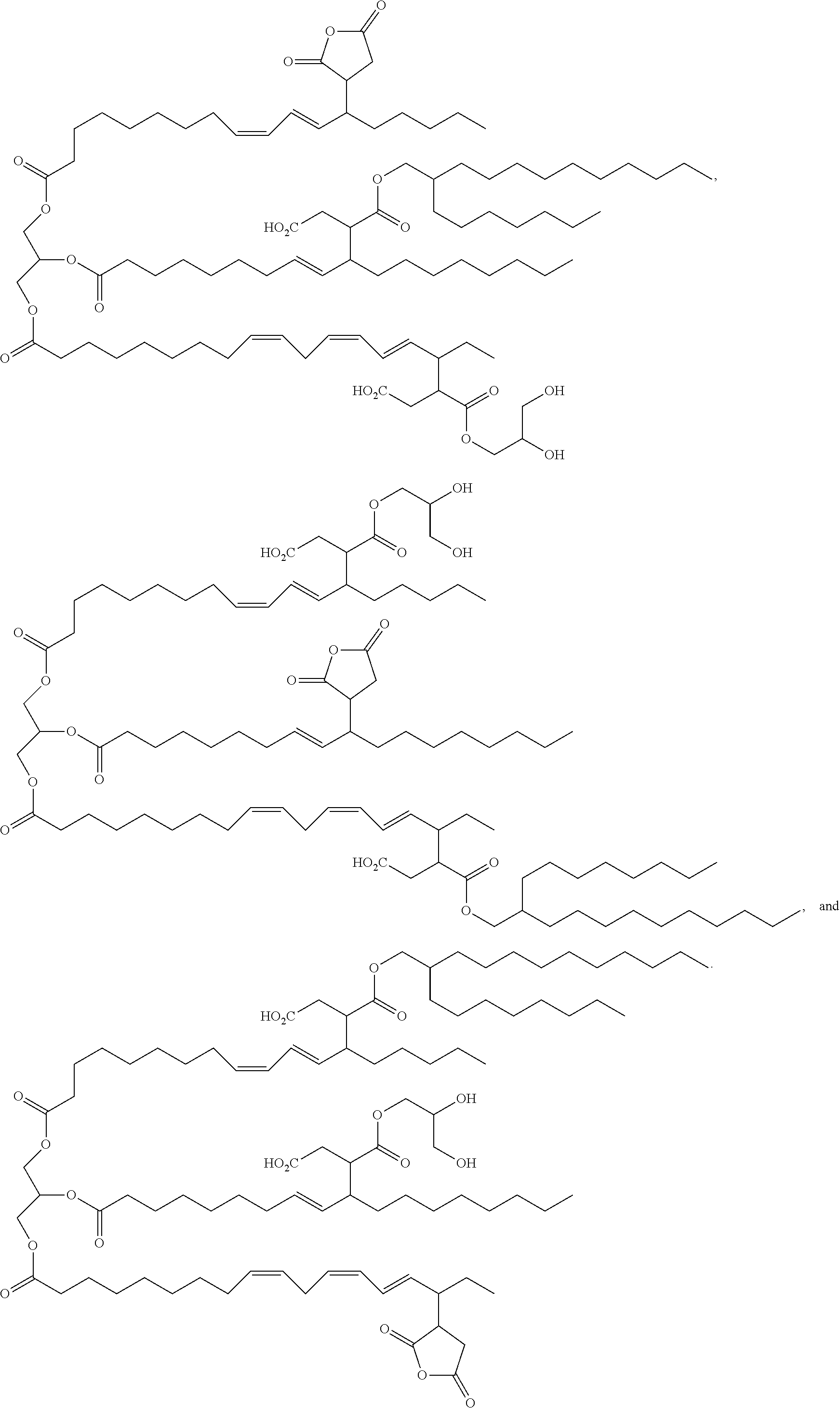

In yet another aspect, the invention provides a personal care composition comprising: (A) a reaction product of: (a) a maleated natural oil, comprising a natural oil with maleated functionality; and (b) a functionalized or unfunctionalized moiety selected from the group consisting of hydrophobic moieties, hydrophilic moieties, and combinations thereof, with the proviso that the hydrophilic moiety is not a glycerol moiety; and (B) (a) a personal care functional system active ingredient.

The preferences listed for the invention set out above for a “composition comprising a reaction product of (a) a maleated natural oil, comprising a natural oil with maleated functionality; and (b) a functionalized or unfunctionalized moiety selected from the group consisting of hydrophobic moieties, hydrophilic moieties, and combinations thereof, with the proviso that the hydrophilic moiety is not a glycerol moiety” also apply to this aspect of the invention.

The composition may be present in an amount of from about 0.01% to about 10% and (B) is present in an amount of from about 90% to about 99.99%.

The personal care functional system active ingredient may be selected from the group consisting of color agents, hair care agents, skin care agents, and sun care agents. In yet another aspect, the invention provides a personal care composition comprising: (A) a reaction product of (a) a maleated natural oil, comprising a natural oil with maleated functionality; (b) a functionalized or unfunctionalized hydrophobic moiety; and (c). a glycerol moiety; and (B) a personal care functional system active ingredient.

The preferences listed for the invention set out above for a “composition comprising: (A) a reaction product of (a) a maleated natural oil, comprising a natural oil with maleated functionality; (b) a functionalized or unfunctionalized hydrophobic moiety; (c) a glycerol moiety; and (B) a functional system active ingredient” also apply to this aspect of the invention.

The composition may be present in an amount of (A) in an amount of from about 0.01% to about 10% and (B) is present in an amount of from about 90% to about 99.99%.

The personal care functional system active ingredient may be selected from the group consisting of color agents, hair care agents, skin care agents, and sun care agents.

The personal care composition may be a formulation selected from the group consisting of sprays, lotions, mousses, fluids, serums, solutions, suspensions, perms, emulsions, gels, mists, vesicles, dispersions, pastes, creams, solid sticks, shampoos, balms, wipes, milks, foams, jellies and liquids.

In yet another aspect, the invention provides a skin care composition comprising: (A) a reaction product of: (a) a maleated natural oil, comprising a natural oil with maleated functionality; and (b) a functionalized or unfunctionalized moiety selected from the group consisting of hydrophobic moieties, hydrophilic moieties, and combinations thereof, with the proviso that the hydrophilic moiety is not a glycerol moiety; and (B)(a) a skin care functional system active ingredient.

The preferences listed for the invention set out above for a “composition comprising: (A) a reaction product of (a) a maleated natural oil, comprising a natural oil with maleated functionality; (b) a functionalized or unfunctionalized hydrophobic moiety; (c) a glycerol moiety; and (B) a functional system active ingredient” also apply to this aspect of the invention.

The composition may be present in an amount of (A) in an amount of from about 0.01% to about 10% and (B) is present in an amount of from about 90% to about 99.99%.

The skin care functional system active ingredient may be selected from the group consisting of color agents, hair care agents, skin care agents, and sun care agents. In yet another aspect, the invention provides a skin care composition comprising: (A) a reaction product of (a) a maleated natural oil, comprising a natural oil with maleated functionality; (b) a functionalized or unfunctionalized hydrophobic moiety; and (c) a glycerol moiety; and (B) a skin care functional system active ingredient.

The preferences listed for the invention set out above for a “composition comprising: (A) a reaction product of (a) a maleated natural oil, comprising a natural oil with maleated functionality; (b) a functionalized or unfunctionalized hydrophobic moiety; (c) a glycerol moiety; and (B) a functional system active ingredient” also apply to this aspect of the invention.

The composition may be present in an amount of (A) in an amount of from about 0.01% to about 10% and (B) is present in an amount of from about 90% to about 99.99%.

Preferably, the skin care functional system active ingredient is selected from the group consisting of color agents, hair care agents, skin care agents, and sun care agents.

Preferably, the skin care functional system active ingredient is a formulation selected from the group consisting of sprays, lotions, mousses, fluids, serums, solutions, suspensions, perms, emulsions, gels, mists, vesicles, dispersions, pastes, creams, solid sticks, shampoos, balms, wipes, milks, foams, jellies and liquids.

In yet another aspect, the present application provides compositions comprising a reaction product of
(a) a maleated natural oil, comprising a natural oil with maleated functionality;
(b) two functionalized or unfunctionalized hydrophilic moieties.

In yet another aspect, the present application provides compositions comprising:
(A) a reaction product of
(a) a maleated natural oil, comprising a natural oil with maleated functionality;
(b) two functionalized or unfunctionalized hydrophilic moieties; and
(B) a functional system active ingredient.

In one non-limiting embodiment, the hydrophobic moiety hydrocarbyl alcohol contains C₂ to C₃₆ carbon atoms and is linear, branched, saturated, unsaturated, aliphatic, aromatic, monofunctional or multifunctional.

In one non-limiting embodiment, the hydrocarbyl alcohol is selected from the group consisting of monohydric alcohols, dihydric alcohols, polyhydric alcohols and combinations thereof.

In one non-limiting embodiment, the hydrocarbyl amine is selected from the group consisting of primary amines, secondary amines and combinations thereof.

Hydrocarbyl amines are classified as primary, secondary and tertiary alcohols, based on the number of carbon atoms connected to the carbon atom that bears the hydroxyl group. Each classification of alcohol may have a general formula. For example,
the general formula for primary amine is

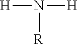

and
the general formula for secondary amine is

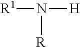

wherein R, R′ stand for hydrogen, different alkyl, alkylene, aryl, aralkyl, arylene, heteroatom groups.

In one non-limiting embodiment, the hydrophobic moiety hydrocarbyl amine contains C₂ to C₃₆ carbon atoms and is linear, branched, saturated, unsaturated, aliphatic, aromatic, monofunctional or multifunctional.

In one non-limiting embodiment, the silicon-base compound is a

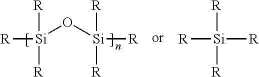

wherein R stands for different alkyl, alkylene, aryl, aralkyl, arylene, hetero groups functionalized with at least one or more alcohol, an amine or a combination thereof and n has the value of 1 to 10.

In one non-limiting embodiment, the silicon-base compound is a siloxane, or a silane functionalized with an alcohol, an amine or a combination thereof.

In one non-limiting embodiment, the silicon-based compound is a linear, branched, saturated, unsaturated, aliphatic, aromatic, monofunctional or multifunctional compound.

In one non-limiting embodiment, the hydrocarbyl alcohol is selected from the group consisting of monohydric alcohols, dihydric alcohols, polyhydric alcohols and combinations thereof.

In one non-limiting embodiment, the hydrocarbyl alcohol is a hydrophobic alcohol selected from the group consisting of propanol, butanol, pentanol, heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol and mixtures thereof.

In one non-limiting embodiment, the hydrocarbyl alcohol is a hydrophilic alcohol selected from the group consisting of methanol, ethanol, propanol, isopropanol, butanol, ethylene glycol, propylene glycol, diethylene glycol, dipropylene glycol, dibutylene glycol, polyethylene glycol, methoxypolyethylene glycol, polypropylene glycol, hexylene glycol, glycerol, sorbitol, octanol, methyl ethyl pentanol, trimethyl pentanol, ethyl hexanol, methyl heptanol, nonanol, methyl octanol, ethyl heptanol, methyl ethyl hexanol, cyclohexanol, dimethyl heptanol, decanol, methyl nonanol, ethyl octanol, trimethyl heptanol, undecanol, methyl decanol, ethyl nonanol, dodecanol, tetradecanol, hexadecanol, octadecanol, benzyl alcohol, phenoxyethanol, neopentylglycol, trimethylol propane, methyldiethanol amine, erythritol, mannitol, xylitol, pentaerythritol, threitol, pentaerythritol, beta-cyclodextrin, L-Ribose, 2-deoxy-D-galactose and mixtures thereof.

In one non-limiting embodiment, the hydrocarbyl amine is hydrophobic amine selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine and mixtures thereof.

In one non-limiting embodiment, the hydrocarbyl amine is hydrophilic amine selected from the group consisting of 2-methylpentane-1,5-diamine, diethanol amine, diisopropanolamine, serinol hydrochloride, 2-amino-2-ethyl-1,3-propanediol, N-methyl-D-glucosamine, D-galactosamine hydrochloride, D-glucosamine hydrochloride, D-mannosamine hydrochloride and mixtures thereof.

In one non-limiting embodiment, the silicon-based compound is a hydrophobic compound selected from the group consisting of aminopropylmethylsiloxane-dimethylsiloxane, N-ethylaminoisobutyl terminated polydimethylsiloxane, poly(1,1-dimethyl silazane) telomer, aminopropyl terminated polydimethylsiloxane, monoaminopropyl terminated polydimethylsiloxane, (tetramethylpiperidinyloxy)propylmethyl siloxane]-dimethyl siloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methyl siloxane and mixtures thereof.

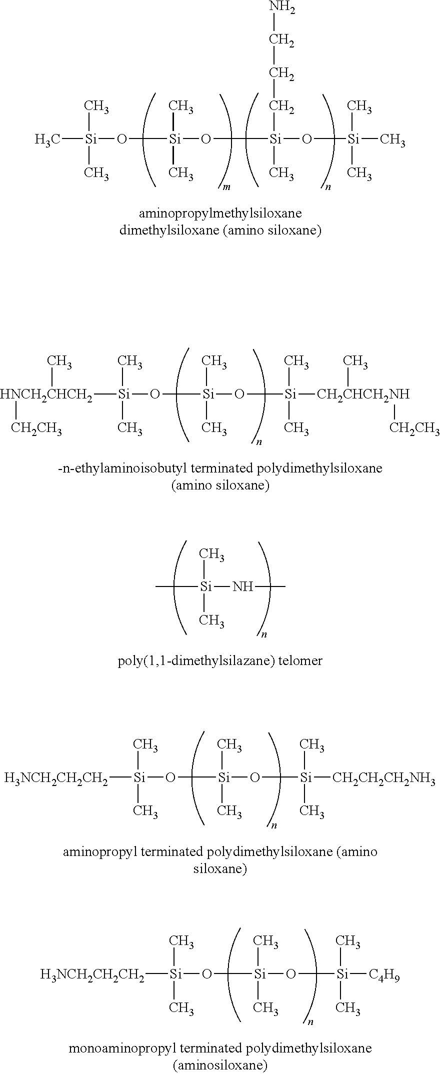

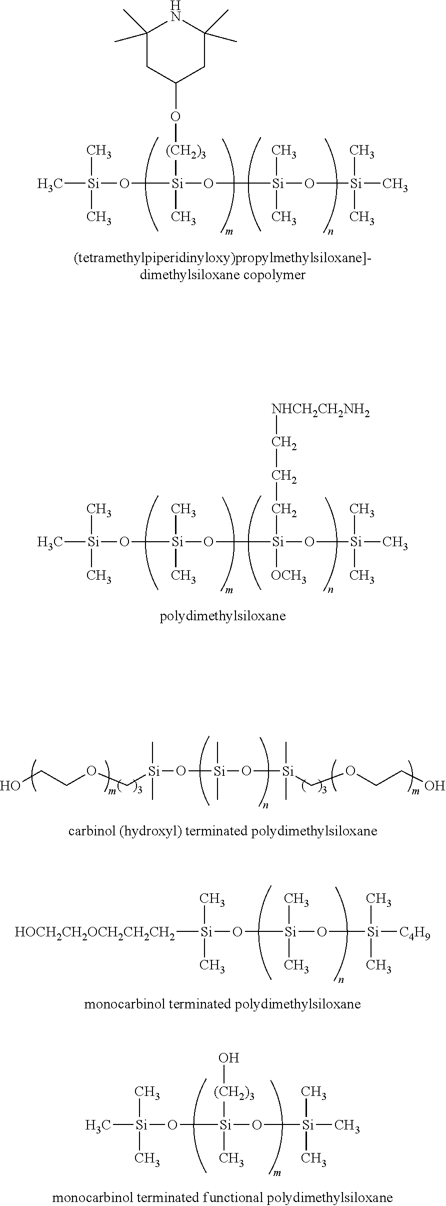

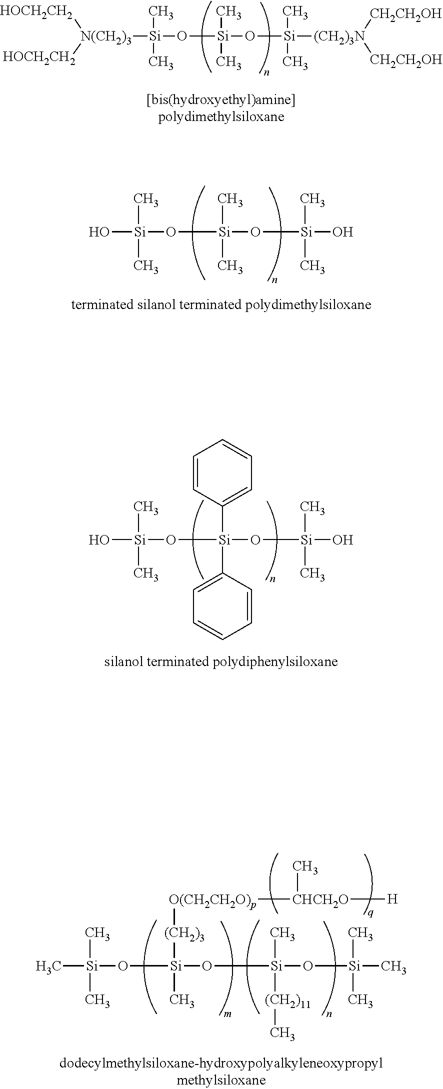

In one non-limiting embodiment, the silicon-based compound is hydrophilic compound selected from the group consisting of 3-aminopropylsilanetriol, N-(2-aminoethyl)-3-aminopropylsilanetriol and mixtures thereof.

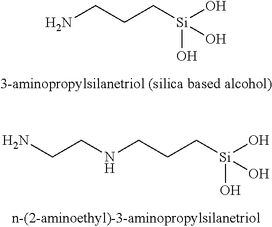 [SMILES: NCCC[Si](O)(O)O ; NCCNCCC[Si](O)(O)O]

In one non-limiting embodiment, the maleated natural oil is selected from the group consisting of a maleated avocado oil, a maleated coconut oil, a maleated corn oil, a maleated cottonseed oil, a maleated jojoba oil, a maleated linseed oil, a maleated nut oil, a maleated olive oil, a maleated palm oil, a maleated raisin oil, a maleated rapeseed oil, a maleated safflower oil, a maleated sesame oil, a maleated soybean oil, a maleated squash oil, a maleated sunflower oil, a maleated almond oil, a maleated canola oil, a maleated flaxseed oil, a maleated grapeseed oil, a maleated palm oil, a maleated palm kernel oil, a maleated peanut oil, and a maleated walnut oil thereof.

The reactions according to the application may be readily synthesized by procedures known by those skilled in the art, non-limiting examples of which include free radical solution polymerization, dispersion polymerization, emulsion polymerization, ionic chain polymerization, living polymerization, bulk polymerization, suspension polymerization or precipitation polymerization. Particularly, the polymerization is carried out by any one of the methods disclosed in “Principles of Polymerization” 4th edition, 2004, Wiley by George Odian and is referred and disclosed herein in its entirety and described in “Decomposition Rate of Organic Free Radical Polymerization” by K. W. Dixon (section II in Polymer Handbook, volume 1,4th edition, Wiley-Interscience, 1999), which is herein incorporated in its entirety by reference.

The maleinization reaction in natural oil can occur under heating in three different ways. The first one is known as “Ene” reaction (reaction between an allylic hydrogen and an enophile-pericyclic reaction), obtaining a triglyceride structure with anhydride moieties (succinic anhydride). The second one is a radical addition, which consumes a double bond in the fatty acid, incorporating the succinic anhydride into the natural oil structure. The final reaction is also a radical addition that incorporates the maleic anhydride into the natural oil structure without consuming C═C bonds (fatty acid chain and maleic anhydride); this reaction occurs due to deprotonation of a hydrogen between two alkenes groups. Subsequently, the maleinized natural oil can be reacted with hydrophobic moiety and the hydrophilic moiety of a hydrocarbyl alcohol, a hydrocarbyl amine, a silicon-based compound forming a reticulated structure (see examples). This kind of reaction has been applied at the reaction occurred by heating 6-10 hours at 210° C.

Thus, the reaction of the non-limiting example above, for illustration purposes only, may be performed at elevated temperatures, such as a temperature between about 150° C. and about 300° C., alternatively between about 170° C. and about 230° C., or alternatively between about 200° C. and about 220° C. The reaction time may be between about 0.5 hours and about 10 hours. In one embodiment the reaction time is between about 1 hour and about 5 hours, and in another embodiment, between about 2 hours and 4 hours. and in another embodiment, between about 6 hours and 10 hours.

During maleation, the mole ratio of natural oil to maleic anhydride in some embodiments is equal to 1, in other embodiments from 1 to 2, in other embodiments from 1 to 2.8 and in still other embodiments from 1 to 3.2 moles of maleic anhydride for each mole of natural oil.

The mole ratio of reaction of maleated natural oil and a hydrophobic moiety, a hydrophilic moiety or a combination thereof mixtures is equal to 1:1 to form a reaction product comprising at least one unreacted maleated functionality.

In one non-limiting embodiment, the composition is a skin care composition, an oral care composition, a hair care composition, an energy composition, a construction composition, a biocidal composition, a preservative composition, a nutraceutical composition, a food composition, an agricultural composition, a coating composition, a cosmetic composition, a homecare composition, an industrial and institutional composition, a textile composition, a laundry composition, a cleaning composition or a disinfecting composition.

According to another embodiment of the present application, it is contemplated to employ at least one functional system active ingredient selected from the group consisting of skin care ingredients, hair care ingredients, oral care ingredients, home care ingredients, energy based ingredients, construction based ingredients, biocide based ingredients, preservative based ingredients, nutraceutical based ingredients, food based ingredients, agricultural based ingredients, coating based ingredients, cosmetic based ingredients, industrial and institutional based ingredients, textile based ingredients, laundry based ingredients, cleaning based ingredients or disinfection based ingredients.

According to another embodiment of the present application, it is contemplated to employ at least one personal care ingredient includes color cosmetic ingredients, hair care ingredients, skin care ingredients or sun care ingredients According to another embodiment of the present application, it is contemplated to employ at least one personal care ingredient selected from the group consisting of water-insoluble ingredients, oxidizing agents, conditioning agents, humectants, pH adjusting buffers, waxes, mineral oils, emulsifiers, fatty substances, gelling agents, thickeners, emollients, hydrophilic or lipophilic active agent, antioxidants, sequestering agents, preserving agents, acidifying or basifying agents, fragrances, fillers, dyestuffs, plant extracts, moisturizers, proteins, peptides, neutralizing agents, solvents, anti-dandruff ingredients, reducing agents and combinations thereof.

In some embodiments, the suitable range of a reaction product of a maleated natural oil and one or more functionalized or unfunctionalized moieties selected from a hydrophobic moiety, a hydrophilic moiety or a combination can be varied from about 0.01 wt. % to about 0.1 wt. %, from about 0.1 wt. % to about 1 wt. %, or from about 1 wt. % to about 5 wt. %, or from about 5 wt. % to about 10 wt. % based on the total weight of the aqueous personal care composition.

In some embodiments, the suitable range of functional system active ingredient for the present application can be varied from about 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. % based on the total weight of the oral care antimicrobial

In some embodiments, the suitable range of personal care ingredient for the present application can be varied from about 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. % based on the total weight of the oral care antimicrobial composition.

In some embodiments, the suitable range of skin care ingredient for the present application can be varied from about 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. % based on the total weight of the oral care antimicrobial composition.

As per another embodiment of the present application, the aqueous personal care composition of the present application is formulated into a spray, a lotion, a mousse, a fluid, a serum, a solution, a perm, an emulsion, a gel, a vesicle a dispersion, a paste, a cream, a solid stick, a shampoo, a balm, a wipe, a milk, a foam, a jelly and/or a liquid.

The reactions and compositions according to the application may be analyzed by known techniques. Especially preferred are the techniques of 13C nuclear magnetic resonance (NMR) spectroscopy, gas chromatography (GC), Infra-red (IR), Liquid Chromatography(LC) and gel permeation chromatography (GPC) in order to decipher identity, residual monomer concentrations, molecular weight, and molecular weight distribution.

Further, certain aspects of the present application are illustrated in detail by way of the following examples. The examples are given herein for illustration of the application and are not intended to be limiting thereof.

### EXAMPLES

### Example A: Grafting of Maleic Anhydride onto Natural Oils

### Example A1: Grafting of 1 Mole of Maleic Anhydride onto Soybean Oil

In a 1-Liter, 4 neck kettle equipped with a thermocouple, a condenser, a nitrogen sparge adapter, and a mechanical stirrer, 600 g of yellow color Soybean oil and 67 g (1 mole eq. based on SBO) of maleic anhydride were charged. The mixture was sparged with nitrogen for 15 min at RT only. The mixture was slowly heated from room temperature to 210° C. and was held isothermally at 210° C. for about 6-8 hours. The one pot reaction without catalyst and solvent yields >96% amber color viscous product which was characterized by NMR and LC<1% residual maleic anhydride.

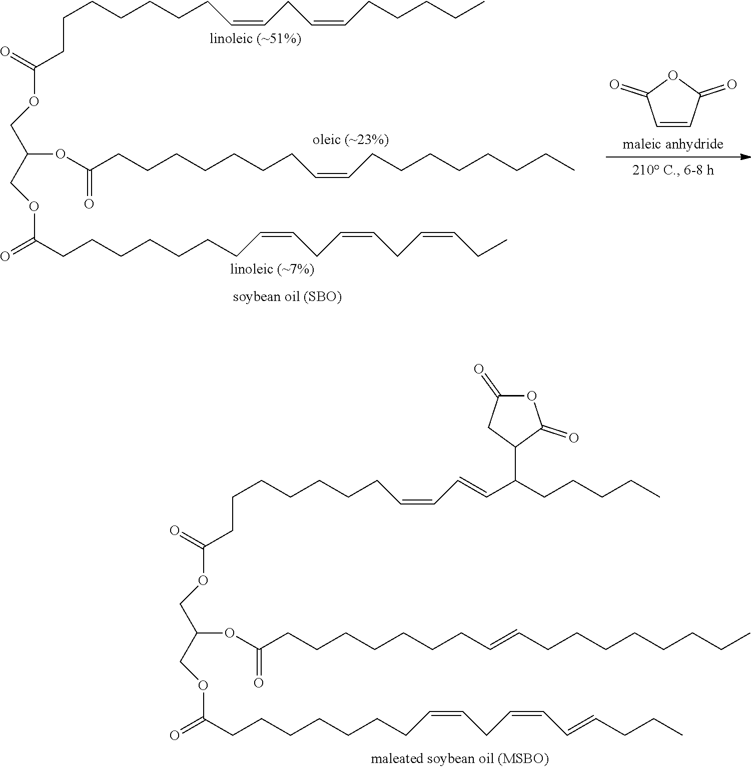 [SMILES: CC/C=C\C/C=C\C/C=C\CCCCCCCC(=O)OCC(COC(=O)CCCCCCC/C=C\C/C=C\CCCCC)OC(=O)CCCCCCC/C=C\CCCCCCCC ; O=C1C=CC(=O)O1 ; CCC/C=C/C=C\C/C=C\CCCCCCCC(=O)OCC(COC(=O)CCCCCCC/C=C\C=C\C(CCCCC)C1CC(=O)OC1=O)OC(=O)CCCCCCC/C=C/CCCCCCCC]

### Example A2: Grafting of 2 Moles of Maleic Anhydride onto Soybean Oil

In a 1-Liter, 4 neck kettle equipped with a thermocouple, a condenser, a nitrogen sparge adapter, and a mechanical stirrer, 600 g of yellow color Soybean oil and 137.7 g (2 mole eq. based on SBO) of maleic anhydride were charged. The mixture was sparged with nitrogen for 15 min at RT only. The mixture was slowly heated from room temperature to 210° C. and was held isothermally at 210° C. for about 6-8 hours. The one pot reaction without catalyst and solvent yields >96% amber color viscous product which was characterized by NMR and LC<1% residual maleic anhydride.

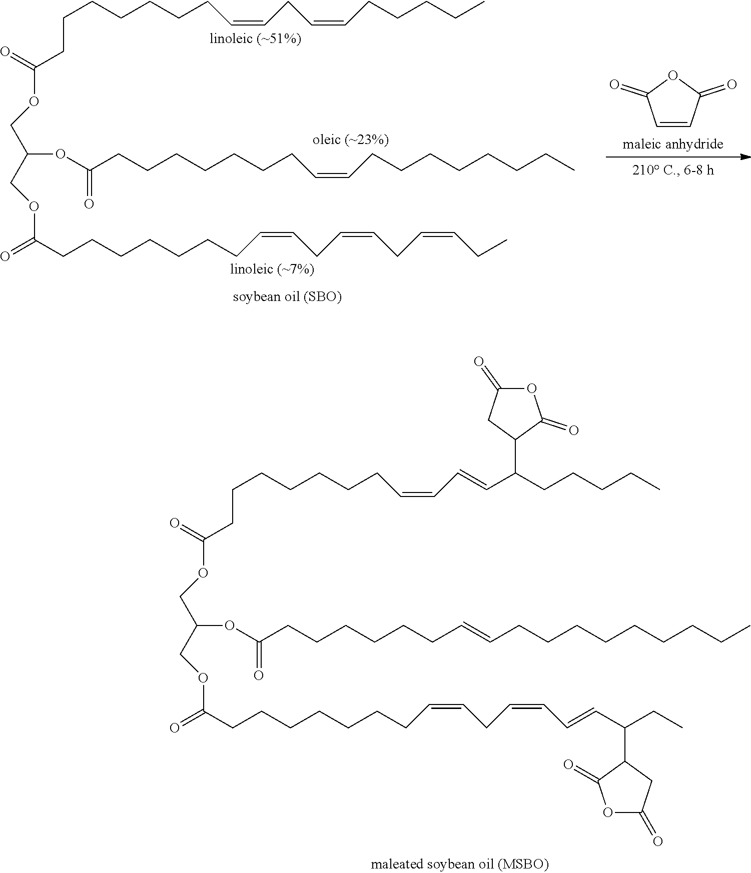 [SMILES: CC/C=C\C/C=C\C/C=C\CCCCCCCC(=O)OCC(COC(=O)CCCCCCC/C=C\C/C=C\CCCCC)OC(=O)CCCCCCC/C=C\CCCCCCCC ; O=C1C=CC(=O)O1 ; CCCCCCCCC/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C1CC(=O)OC1=O]

### Example A3: Grafting of 3 Moles of Maleic Anhydride onto Soybean Oil

In a 1-Liter, 4 neck kettle equipped with a thermocouple, a condenser, a nitrogen sparge adapter, and a mechanical stirrer, 600 g of yellow color soybean oil and 204 g (3 mole eq. based on SBO) of maleic anhydride were charged. The mixture was sparged with nitrogen for 15 min at RT only. The mixture was slowly heated from room temperature to 210° C. and was held isothermally at 210° C. for about 6-8 hours. The one pot reaction without catalyst and solvent yields >96% amber color viscous product which was characterized by NMR and LC<1% residual maleic anhydride

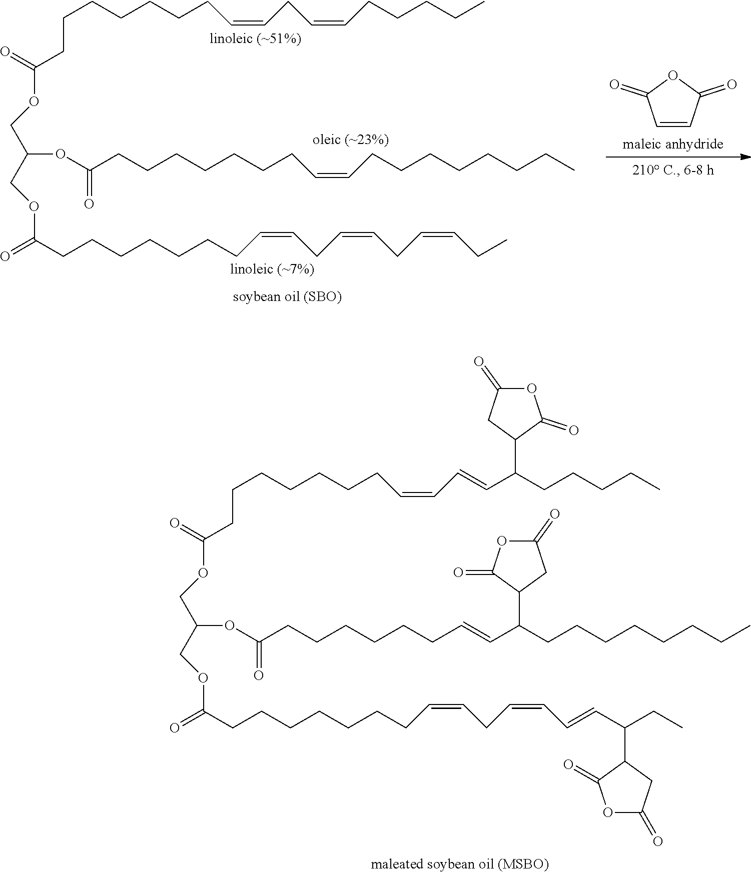 [SMILES: CC/C=C\C/C=C\C/C=C\CCCCCCCC(=O)OCC(COC(=O)CCCCCCC/C=C\C/C=C\CCCCC)OC(=O)CCCCCCC/C=C\CCCCCCCC ; O=C1C=CC(=O)O1 ; CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C1CC(=O)OC1=O)C1CC(=O)OC1=O]

### Example A4: Grafting of Maleic Anhydride onto Palm Oil

In a 1-Liter, 4 neck kettle equipped with a thermocouple, a condenser, a nitrogen sparge adapter, and a mechanical stirrer, 100 g of palm oil and 23 g (2 mole eq. based on palm oil) of maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber color viscous product which was characterized by NMR and LC<1% residual maleic anhydride.

### Example A5: Grafting of Maleic Anhydride onto Canola Oil

In a 1-Liter, 4 neck kettle equipped with a thermocouple, a condenser, a nitrogen sparge adapter, and a mechanical stirrer, 100 g of canola oil and 22.2 g (2 mole eq. based on canola oil) of maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber color viscous product which was characterized by NMR and LC<1% residual maleic anhydride.

### Example A6: Grafting of Maleic Anhydride onto Sunflower Oil

In a 1-Liter, 4 neck kettle equipped with a thermocouple, a condenser, a nitrogen sparge adapter, and a mechanical stirrer, 100 g of sunflower oil and 22.4 g (2 mole eq. based on sunflower oil) of maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber color viscous product which was characterized by NMR and LC<1% residual maleic anhydride.

### Example A7: Grafting of Maleic Anhydride onto Castor Oil

In a 1-Liter, 4 neck kettle equipped with a thermocouple, a condenser, a nitrogen sparge adapter, and a mechanical stirrer, 100 g of yellow color castor oil and 21 g (2 mole eq. based on castor oil) of maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber color viscous product which was characterized by NMR and LC<1% residual maleic anhydride.

### Example B: Grafting of Hydrophobic Alcohol (Octyl Dodecanol) onto Maleic Anhydride onto Natural Oil

### Example B1

In a 1-Liter, 4 neck kettle equipped with a thermocouple, a condenser, a nitrogen purge adapter, and a mechanical stirrer, 100 g of amber color viscous product from maleination reaction Example A1 and 30.5 g (1 mole eq.) of 2-octyl-1-dodecanol were mixed and heated to 90° C. and held for 6 hours. The one pot reaction without catalyst and solvent yields >96% amber color viscous product, which was characterized by NMR, IR, GC<5% for residual octyl dodecanol and LC<1% residual maleic anhydride.

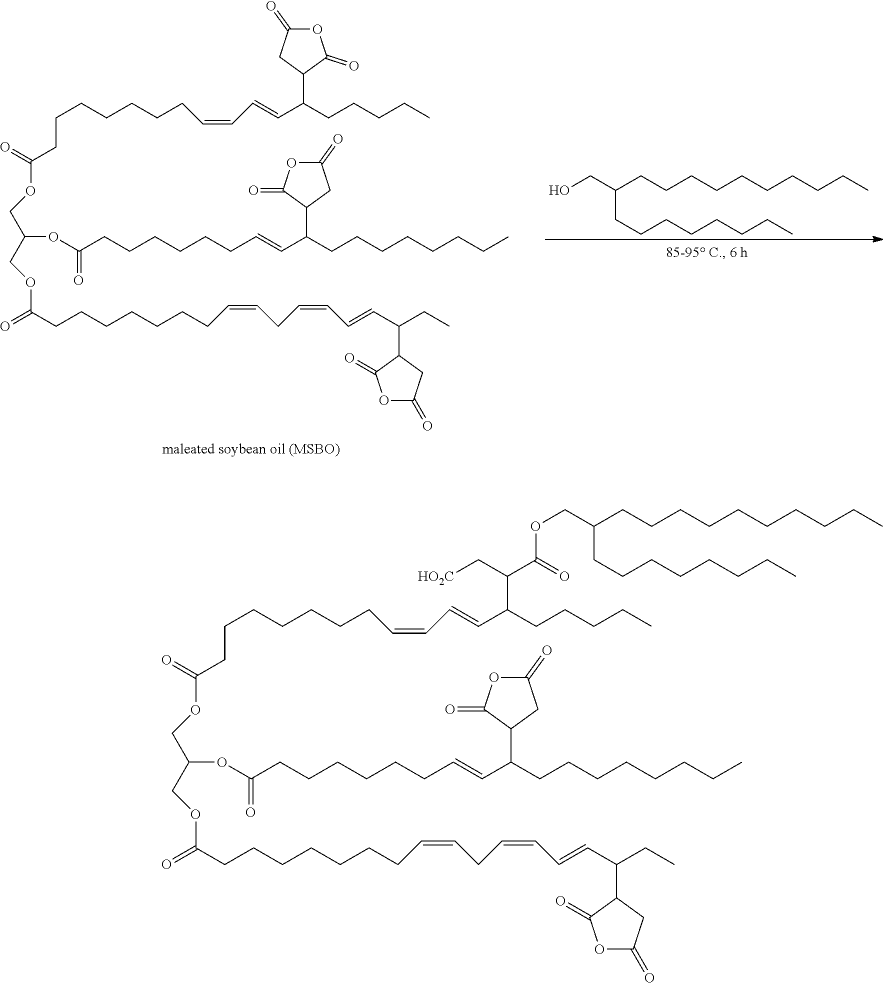 [SMILES: CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C1CC(=O)OC1=O)C1CC(=O)OC1=O ; CCCCCCCCCCC(CO)CCCCCCCC ; CCCCCCCCCCC(CCCCCCCC)COC(=O)C(CC(=O)O)C(/C=C/C=C\CCCCCCCC(=O)OCC(COC(=O)CCCCCCC/C=C\C/C=C\C=C\C(CC)C1CC(=O)OC1=O)OC(=O)CCCCCC/C=C/C(CCCCCCCC)C1CC(=O)OC1=O)CCCCC]

### Example B2

In a 1-Liter, 4 neck kettle equipped with a thermocouple, a condenser, a nitrogen purge adapter, and a mechanical stirrer, 100 g of amber color viscous product from maleination reaction Example A2 and 55.64 g (2 mole eq.) of 2-octyl-1-dodecanol were mixed and heated to 90° C. and held for 6 hours. The one pot reaction without catalyst and solvent yields >96% amber color viscous product, which was characterized by NMR, IR, GC<5% for residual octyl dodecanol and LC<1% residual maleic anhydride.

### Example B3

In a 1-Liter, 4 neck kettle equipped with a thermocouple, a condenser, a nitrogen purge adapter, and a mechanical stirrer, 100 g of amber color viscous product from maleination reaction Example A2 and 61.20 g (2.2 mole eq.) of 2-octyl-1-dodecanol were mixed and heated to 90° C. and held for 6 hours. The one pot reaction without catalyst and solvent yields >96% amber color viscous product which was characterized by NMR, GC<5% for residual octyl dodecanol and LC<1% residual maleic anhydride.

### Example B4

In a 1-Liter, 4 neck kettle equipped with a thermocouple, a condenser, a nitrogen purge adapter, and a mechanical stirrer, 100 g of amber color viscous product from maleination reaction Example A3 and 25.44 g (1 mole eq.) of 2-octyl-1-dodecanol were mixed and heated to 90° C. and held for 6 hours. The one pot reaction without catalyst and solvent yields >96% amber color viscous product, which was characterized by NMR, IR, GC<5% for residual octyl dodecanol and LC<1% residual maleic anhydride.

### Example B5

In a 1-Liter, 4 neck kettle equipped with a thermocouple, a condenser, a nitrogen purge adapter, and a mechanical stirrer, 100 g of amber color viscous product from maleination reaction Example A3 and 31.81 g (1.25 mole eq.) of 2-octyl-1-dodecanol were mixed and heated to 90° C. and held for 6 hours. The one pot reaction without catalyst and solvent yields >96% amber color viscous product, which was characterized by NMR, IR, GC<5% for residual octyl dodecanol and LC<1% residual maleic anhydride.

### Example B6

In a 1-Liter, 4 neck kettle equipped with a thermocouple, a condenser, a nitrogen purge adapter, and a mechanical stirrer, 100 g of amber color viscous product from maleination reaction Example A3 and 38.17 g (1.5 mole eq.) of 2-octyl-1-dodecanol were mixed and heated to 90° C. and held for 6 hours. The one pot reaction without catalyst and solvent yields >96% amber color viscous product, which was characterized by NMR, IR, GC<5% for residual octyl dodecanol and LC<1% residual maleic anhydride.

### Example B7

In a 1-Liter, 4 neck kettle equipped with a thermocouple, a condenser, a nitrogen purge adapter, and a mechanical stirrer, 100 g of amber color viscous product from maleination reaction Example A 3 and 50.89 g (2 mole eq.) of 2-octyl-1-dodecanol were mixed and heated to 90° C. and held for 6 hours. The one pot reaction without catalyst and solvent yields >96% amber color viscous product, which was characterized by NMR, IR, GC<5% for residual octyl dodecanol and LC<1% residual maleic anhydride.

### Example B8

In a 1-Liter, 4 neck kettle equipped with a thermocouple, a condenser, a nitrogen purge adapter, and a mechanical stirrer, 100 g of amber color viscous product from maleination reaction Example A3 and 63.61 g (2.5 mole eq.) of 2-octyl-1-dodecanol were mixed and heated to 90° C. and held for 6 hours. The one pot reaction without catalyst and solvent yields >96% amber color viscous product, which was characterized by NMR, IR, GC<5% for residual octyl dodecanol and LC<1% residual maleic anhydride.

### Example B9

In a 1-Liter, 4 neck kettle equipped with a thermocouple, a condenser, a nitrogen purge adapter, and a mechanical stirrer, 100 g of amber color viscous product from maleination reaction Example A4 and 35 g (1 mole eq.) of 2-octyl-1-dodecanol was mixed and heated to 90° C. and held for 6 hours. The one pot reaction without catalyst and solvent yields >96% amber color viscous product, which was characterized by NMR, IR, GC<5% for residual octyl dodecanol and LC<1% residual maleic anhydride.

### Example B10

In a 1-Liter, 4 neck kettle equipped with a thermocouple, a condenser, a nitrogen purge adapter, and a mechanical stirrer, 100 g of amber color viscous product from maleination reaction Example A5 and 35 g (1 mole eq.) of 2-octyl-1-dodecanol was mixed and heated to 90° C. and held for 6 hours. The one pot reaction without catalyst and solvent yields >96% amber color viscous product, which was characterized by NMR, IR, GC<5% for residual octyl dodecanol and LC<1% residual maleic anhydride.

### Example B11

In a 1-Liter, 4 neck kettle equipped with a thermocouple, a condenser, a nitrogen purge adapter, and a mechanical stirrer, 100 g of amber color viscous product from maleination reaction Example A6 and 35 g (1 mole eq.) of 2-octyl-1-dodecanol was mixed and heated to 90° C. and held for 6 hours. The one pot reaction without catalyst and solvent yields >96% amber color viscous product, which was characterized by NMR, IR, GC<5% for residual octyl dodecanol and LC<1% residual maleic anhydride.

### Example B12

In a 1-Liter, 4 neck kettle equipped with a thermocouple, a condenser, a nitrogen purge adapter, and a mechanical stirrer, 100 g of amber color viscous product from maleination reaction Example A7 and 35 g (1 mole eq.) of 2-octyl-1-dodecanol was mixed and heated to 90° C. and held for 6 hours. The one pot reaction without catalyst and solvent yields >96% amber color viscous product, which was characterized by NMR, IR, GC<5% for residual octyl dodecanol and LC<1% residual maleic anhydride.

### Example C: Grafting of Hydrophobic and Hydrophilic Moiety (Glycerin) onto Hydrophobic Maleic Anhydride onto Natural Oil with Octyl Dodecanol

### Example C1

In a 1-Liter, 4 neck kettle equipped with a thermocouple, a condenser, a nitrogen purge adapter, and a mechanical stirrer, 100 g of amber color viscous product from reaction B 6 and 5.7 g (1 mole eq.) of glycerin were mixed and heated to 90° C. and held for 4-6 hours. The one pot reaction without catalyst and solvent yields >96% amber color viscous product which was characterized by NMR and LC<0.05% residual maleic anhydride.

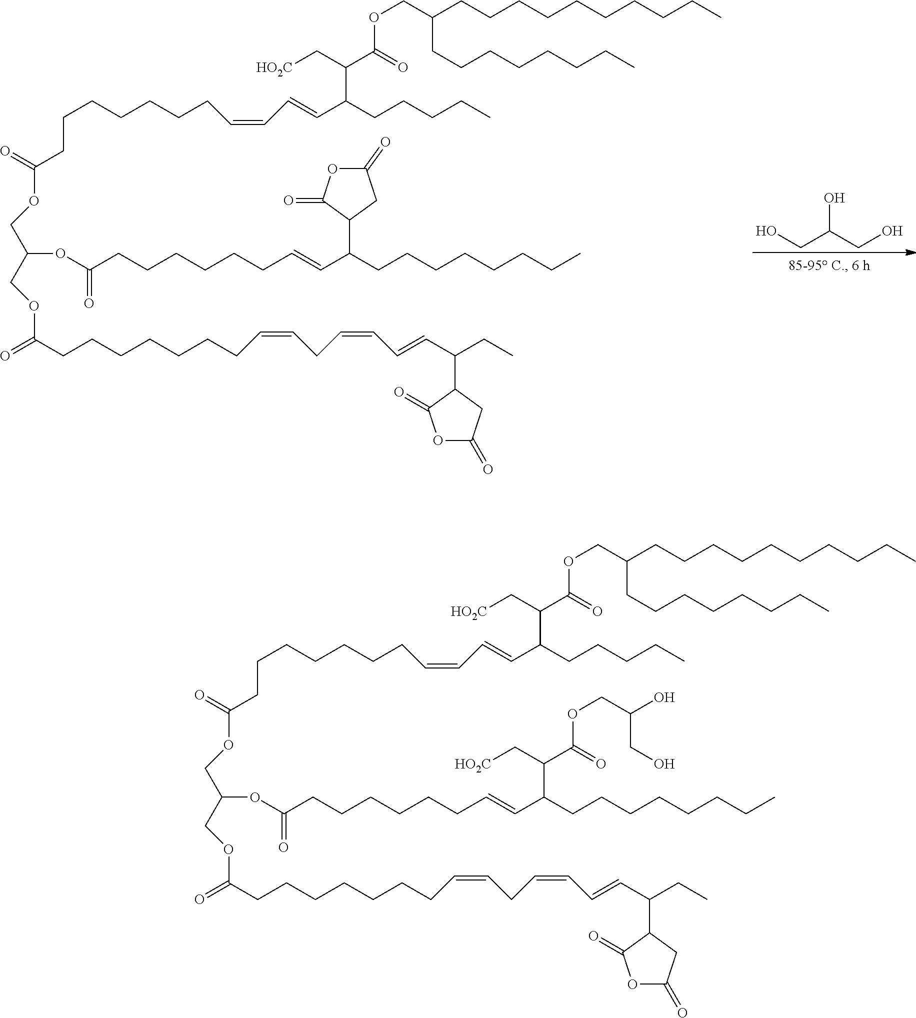 [SMILES: CCCCCCCCCCC(CCCCCCCC)COC(=O)C(CC(=O)O)C(/C=C/C=C\CCCCCCCC(=O)OCC(COC(=O)CCCCCCC/C=C\C/C=C\C=C\C(CC)C1CC(=O)OC1=O)OC(=O)CCCCCC/C=C/C(CCCCCCCC)C1CC(=O)OC1=O)CCCCC ; OCC(O)CO ; CCCCCCCCCCC(CCCCCCCC)COC(=O)C(CC(=O)O)C(/C=C/C=C\CCCCCCCC(=O)OCC(COC(=O)CCCCCCC/C=C\C/C=C\C=C\C(CC)C1CC(=O)OC1=O)OC(=O)CCCCCC/C=C/C(CCCCCCCC)C(CC(=O)O)C(=O)OCC(O)CO)CCCCC]

### Example C 2

In a 1-Liter, 4 neck kettle equipped with a thermocouple, a condenser, a nitrogen purge adapter, and a mechanical stirrer, 100 g of amber color viscous product from reaction B 4 and 6.28 g (1 mole eq.) of glycerin were mixed and heated to 90° C. and held for 4-6 hours. The one pot reaction without catalyst and solvent yields >96% amber color viscous product which was characterized by NMR and LC<0.05% residual maleic anhydride.

### Example C 3

In a 1-Liter, 4 neck kettle equipped with a thermocouple, a condenser, a nitrogen purge adapter, and a mechanical stirrer, 100 g of amber color viscous product from reaction B 4 and 9.42 g (1.5 mole eq.) of glycerin were mixed and heated to 90° C. and held for 4 hours. The one pot reaction without catalyst and solvent yields >96% amber color viscous product which was characterized by NMR and LC<0.05% residual maleic anhydride.

### Example C 4

In a 1-Liter, 4 neck kettle equipped with a thermocouple, a condenser, a nitrogen purge adapter, and a mechanical stirrer, 100 g of amber color viscous product from reaction B 9 and 11 g (1 mole eq.) of glycerin was mixed and heated to 90° C. and held for 4-6 hours. The one pot reaction without catalyst and solvent yields >96% amber color viscous product which was characterized by NMR and LC<0.05% residual maleic anhydride.

### Example C 5

In a 1-Liter, 4 neck kettle equipped with a thermocouple, a condenser, a nitrogen purge adapter, and a mechanical stirrer, 100 g of amber color viscous product from reaction B 10 and 11 g (1 mole eq.) of glycerin was mixed and heated to 90° C. and held for 4-6 hours. The one pot reaction without catalyst and solvent yields >96% amber color viscous product which was characterized by NMR and LC<0.05% residual maleic anhydride.

### Example C 6

In a 1-Liter, 4 neck kettle equipped with a thermocouple, a condenser, a nitrogen purge adapter, and a mechanical stirrer, 100 g of amber color viscous product from reaction B 11 and 11 g (1 mole eq.) of glycerin was mixed and heated to 90° C. and held for 4-6 hours. The one pot reaction without catalyst and solvent yields >96% amber color viscous product which was characterized by NMR and LC<0.05% residual maleic anhydride.

### Example C 7

In a 1-Liter, 4 neck kettle equipped with a thermocouple, a condenser, a nitrogen purge adapter, and a mechanical stirrer, 100 g of amber color viscous product from reaction B 12 and 11 g (1 mole eq.) of glycerin was mixed and heated to 90° C. and held for 4-6 hours. The one pot reaction without catalyst and solvent yields >96% amber color viscous product which was characterized by NMR and LC<0.05% residual maleic anhydride.

### Example C 8

In a 1-Liter, 4 neck kettle equipped with a thermocouple, a condenser, a nitrogen purge adapter, and a mechanical stirrer, 152 g of amber color viscous product from reaction B 4 and 4.74 g (1 mole eq.) of ethanol were mixed and heated to 90° C. and held for 8 hours. The one pot reaction without catalyst and solvent yields >96% amber color product which was characterized by NMR and LC<0.05% residual maleic anhydride.

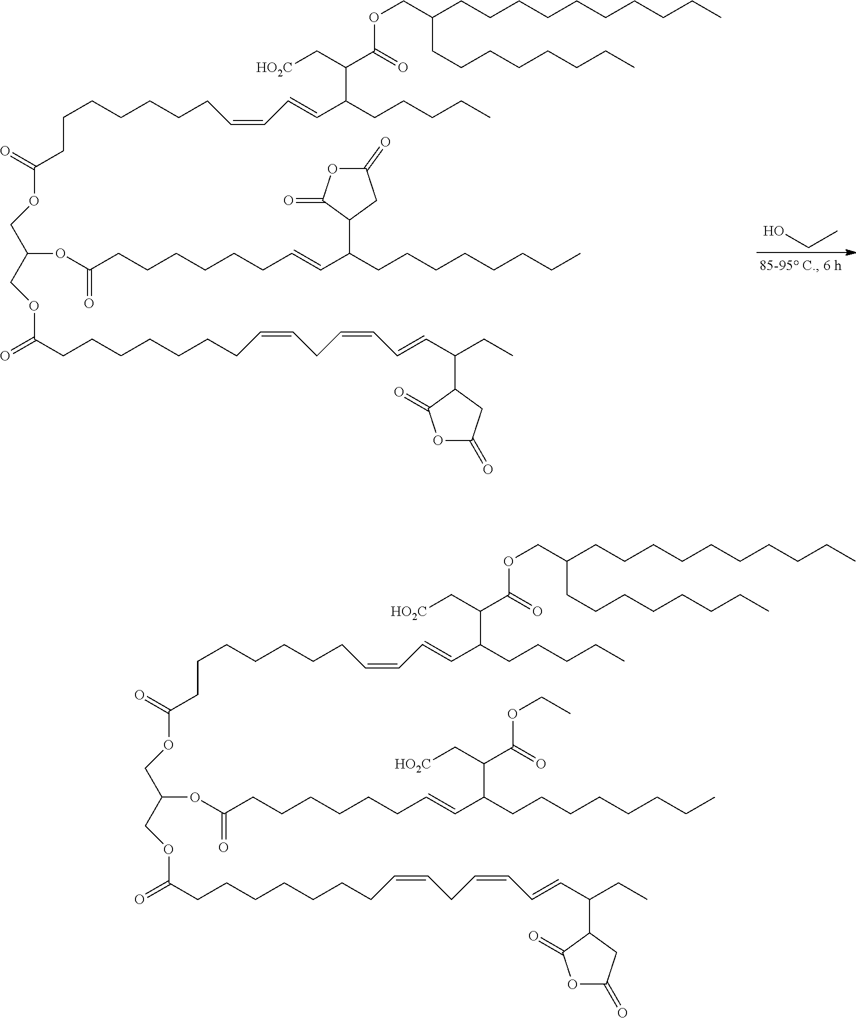

### Example C 9

In a 1-Liter, 4 neck kettle equipped with a thermocouple, a condenser, a nitrogen purge adapter, and a mechanical stirrer, 152 g of amber color viscous product from reaction B 4 and 9.47 g (2 mole eq.) of ethanol were mixed and heated to 90° C. and held for 8 hours. The one pot reaction without catalyst and solvent yields >96% amber color product which was characterized by NMR and LC<0.05% residual maleic anhydride.

### Example D: Grafting of Hydrophilic Alcohol (Beta Cyclodextrin, Glycerin) onto Maleic Anhydride onto Natural Oil

### Example D 1

In a 1-Liter, 4 neck kettle equipped with a thermocouple, a condenser, a nitrogen purge adapter, and a mechanical stirrer, 68.43 g of amber color viscous product from reaction B 6 and 23.73 g (0.50 mole eq.) of beta cyclodextrin were mixed and heated to 90° C. and held for 6 hours. The one pot reaction without catalyst and solvent yields >96% yellow color product which was characterized by IR and LC<0.05% residual maleic anhydride.

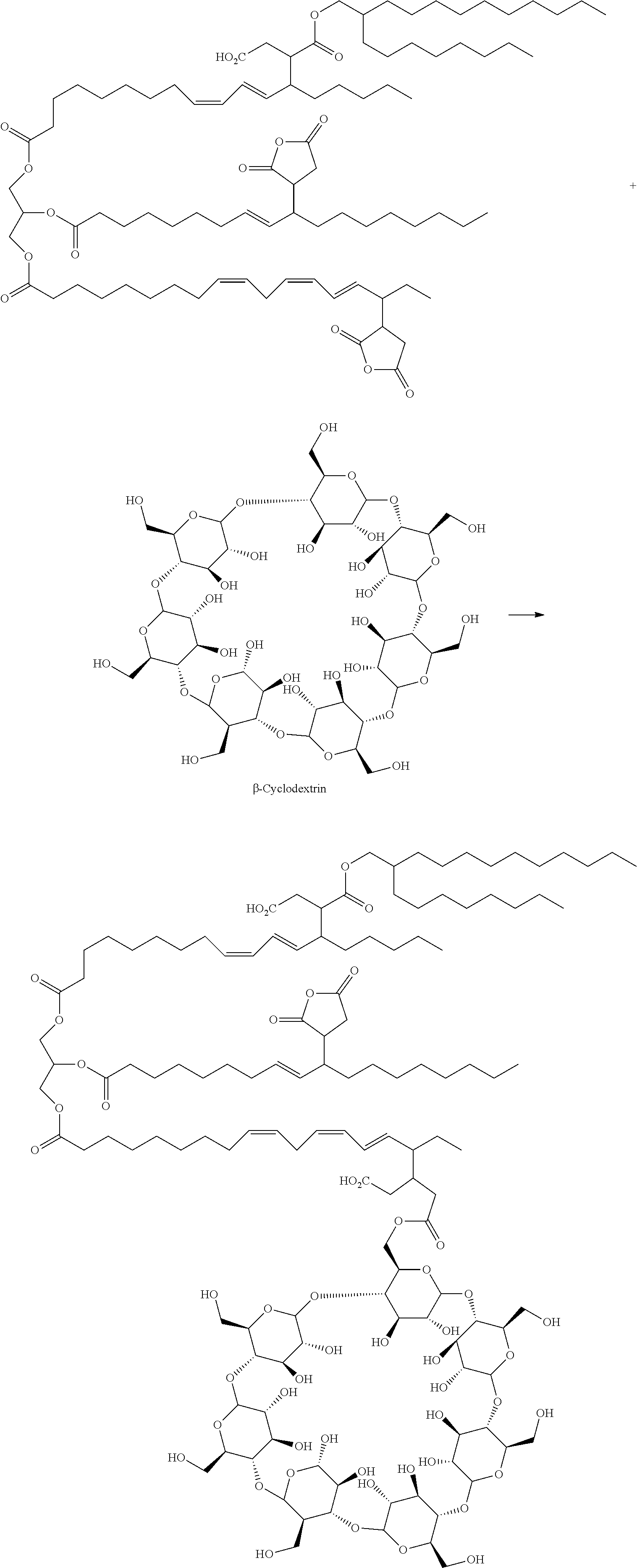

### Example D 2

In a 1-Liter, 4 neck kettle equipped with a thermocouple, a condenser, a nitrogen purge adapter, and a mechanical stirrer, 68.43 g of amber color viscous product from reaction B 6 and 23.73 g (0.50 mole eq.) of beta cyclodextrin and 3.85 g (1 mole eq.) of glycerin were mixed and heated to 90° C. and held for 4 hours. The one pot reaction without catalyst and solvent yields >96% yellow color product which was characterized by IR and LC<0.05% residual maleic anhydride.

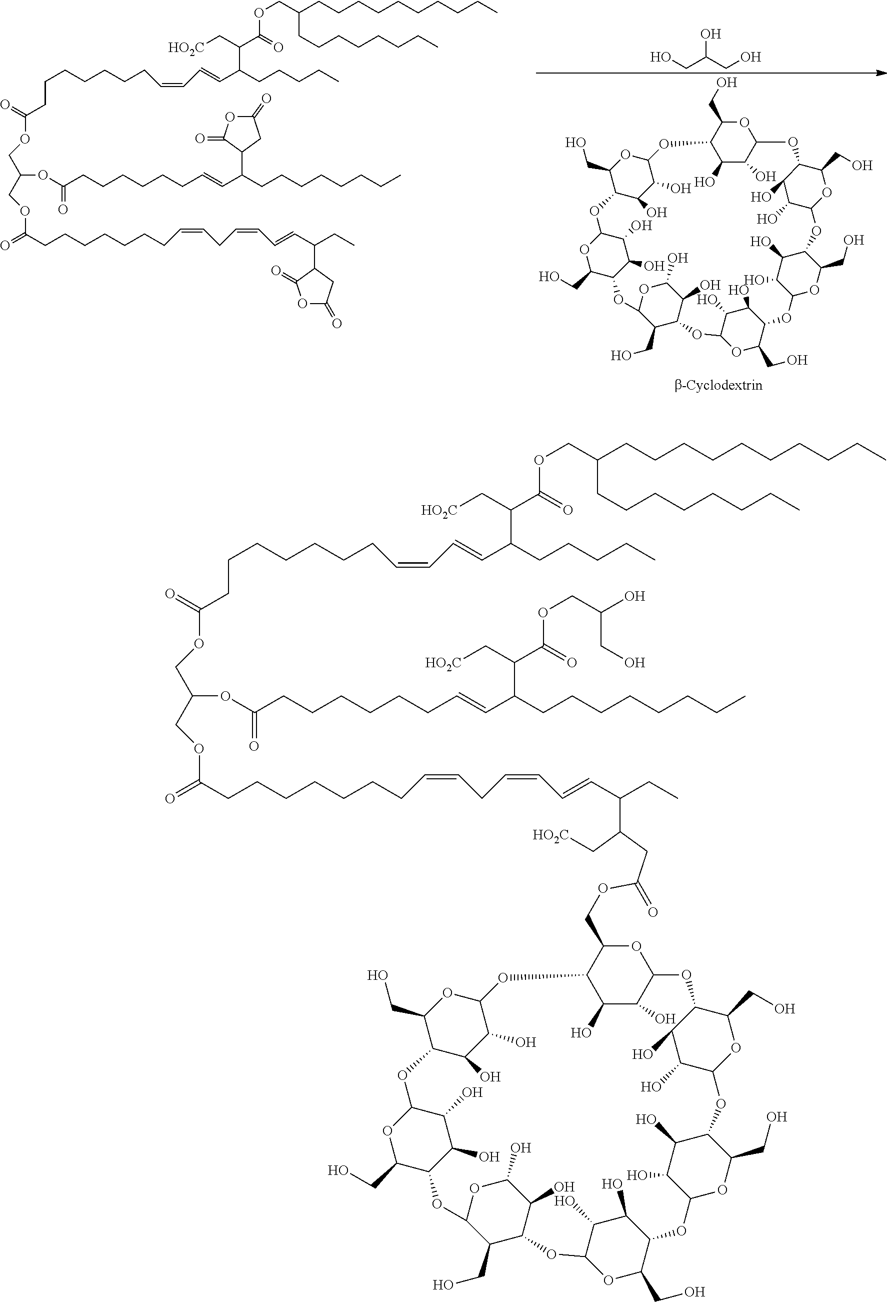

### Example D 3

In a 1-Liter, 4 neck kettle equipped with a thermocouple, a condenser, a nitrogen purge adapter, and a mechanical stirrer, 68.43 g of amber color viscous product from reaction B 6 and 23.73 g (0.50 mole eq.) of beta cyclodextrin and 1.93 g (0.50 mole eq.) of glycerin were mixed and heated to 90° C. and held for 4 hours. The one pot reaction without catalyst and solvent yields >96% yellow color product which was characterized by IR and LC<0.05% residual maleic acid. The one pot reaction without catalyst and solvent yields >96% yellow color product which was characterized by IR and LC<0.05% residual maleic anhydride.

### Example E: Maleination Reaction with Water

### Example E 1

In a 1-Liter, 4 neck kettle equipped with a thermocouple, a condenser, a nitrogen purge adapter, and a mechanical stirrer, 100 g of amber color viscous product from maleination reaction A 3 and 6.10 g (3 mole eq.) of water were mixed and heated to 90° C. and held for 20 hours. The one pot reaction without catalyst and solvent yields >90% yellow color product which was characterized by NMR, IR and LC<0.05% residual maleic acid.

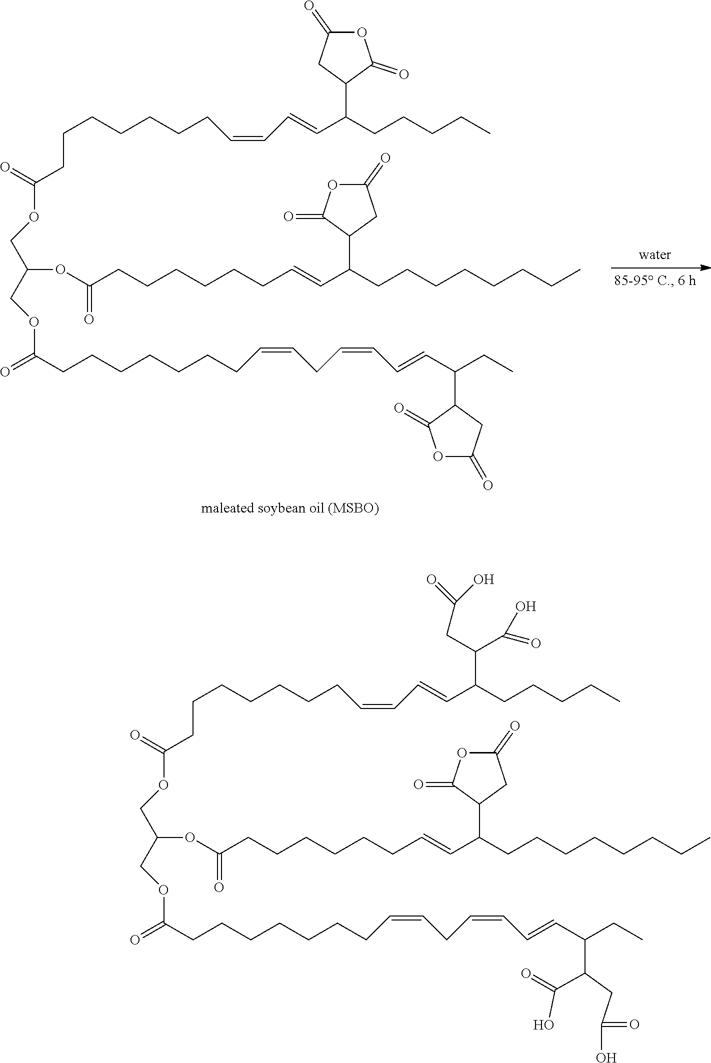 [SMILES: CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C1CC(=O)OC1=O)C1CC(=O)OC1=O ; CCCCCCCCC(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C=C\C(CC)C(CC(=O)O)C(=O)O)C1CC(=O)OC1=O]

### Example F: Grafting of Hydrophilic Glycerin onto Maleic Anhydride onto Soybean Oil

### Example F 1

In a 1-Liter, 4 neck kettle equipped with a thermocouple, a condenser, a nitrogen purge adapter, and a mechanical stirrer, 176 g of amber color viscous product from maleination reaction A 3 and 6.87 g (0.5 mole eq.) of glycerin were mixed and heated to 90° C. and held for 2 hours. The one pot reaction without catalyst and solvent yields >96% yellow color product which was characterized by IR and LC<0.05% residual maleic anhydride.

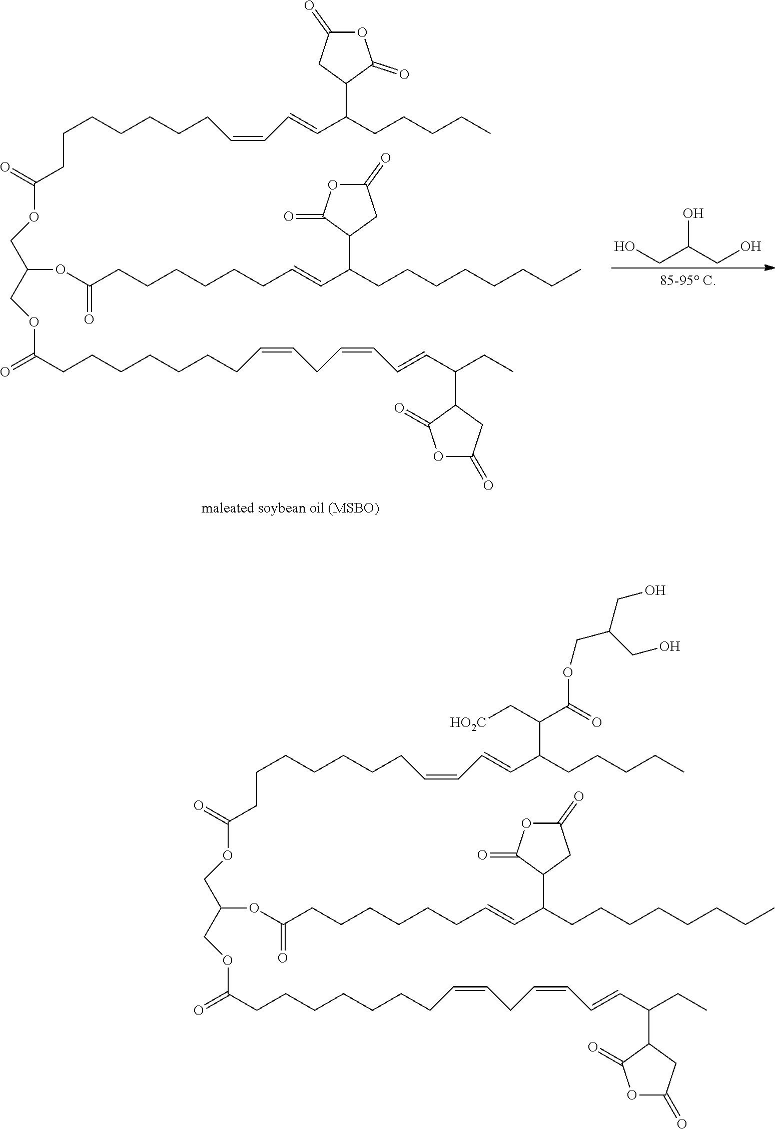 [SMILES: CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C1CC(=O)OC1=O)C1CC(=O)OC1=O ; OCC(O)CO ; CCCCCCCCC(/C=C/CCCCCCC(=O)OC(COC(=O)CCCCCCC/C=C\C=C\C(CCCCC)C(CC(=O)O)C(=O)OCC(CO)CO)COC(=O)CCCCCCC/C=C\C/C=C\C=C\C(CC)C1CC(=O)OC1=O)C1CC(=O)OC1=O]

### Example F 2

In a 1-Liter, 4 neck kettle equipped with a thermocouple, a condenser, a nitrogen purge adapter, and a mechanical stirrer, 176 g of amber color viscous product from maleination reaction A 3 and 13.74 g (1 mole eq.) of glycerin were mixed and heated to 90° C. and held for 4 hours. The one pot reaction without catalyst and solvent yields >96% yellow color product which was characterized by IR and LC<0.05% residual maleic anhydride.

### Nonionic Sunscreen Emulsion Procedure

In Phase A, the water and glycol were added into a glass beaker. Sprinkle in Carbomer, mix until all in and smooth. Weigh out the Phase B ingredients and heated at a temperature of 75-80° C. and was added slowly to the phase A and continue to homogenize. The homogenize mixture was cooled at 60-65° C. Weigh out the Phase C ingredients and slowly add to the homogenize mixture of Phase A and Phase B and continue to homogenize. The pH was adjusted as needed. Sweep mix until the reactor vessel was cooled.

### Water in Silicone Mineral (Zinc and Titanium) Emulsion Procedure

Pre-weigh ingredients of phase A. and the ingredients were mixed and heat to 40-45° C., homogenize until uniform and then cool down at 30-35° C. In a separate beaker phase C were added and heated to 65° C. and continue mix until clear. Cool down the phase C to 35-40° C. Pulverize phase B and add to phase C while mixing at moderate high speed. Add phase A to the mixture of Phase B and C slowly and continue mixing at moderate to high speed for about 10 minutes. Cool to room temperature.

### W/Si Mineral (Zinc) Emulsion Procedure

In Phase A, Dimethicone 1 and Dimethicone 2 were added to main beaker and begin mixing with a high lift mixing blade at high speed. Cetyl PEG/PPG-10/1 Dimethicone was added and continue mixing at high speed then add polyglyceryl-4 isostearate, cetyl PEG/PPG-10/1 Dimethicone and hexyl laurate and continue mixing. Then add the caprylic/capric Triglyceride, stearalkonium hectorite, Propylene Carbonate. In a separate beaker combine Phase B ingredients and mix. Once Phase B mixture was uniform, it was added to phase A and continue mixing. In a separate beaker phase C ingredient were added. Water was added and continue mixing, then hydroxyethyl cellulose was added and heat to 80-85° C. once dispersed, glycerin and butylene glycol were added and continue mixing and heating to 85° C. until the hydroxyethyl cellulose is solubilized and then phenoxyethanol and salt were added. Ensure that all the salt is dissolved and begin cooling slowly to room temperature. Once all the ingredients in phase C was uniform increase mixing on phase A and slowly pour phase C into phase A. Continue mixing until uniform. Zinc Oxide was added to the main batch and continue prop mixing for 5 minutes until the batch is homogenous. Switch to the homogenizer and continue homogenizing for 10 minutes.

### Anionic Emulsion Procedure

In Phase A, water and Glycerin and Glyceryl Acrylate/Acrylic Acid Copolymer and PVM/MA copolymer were added into a glass beaker and heated to 75-80° C. Sprinkle in Acrylic Acid/VP crosspolymer, mix until all in and smooth. Weigh out the Phase B ingredients and heated at a temperature of 75-80° C. and was added slowly to the phase A and continue to homogenize. The homogenize mixture was cooled at 60-65° C. Weigh out the Phase C ingredients and slowly add to the homogenize mixture of Phase A and Phase B and continue to homogenize. The pH was adjusted as needed. Sweep mix until the reactor vessel was cooled.

### Hydrosheer™ Spray Procedure

Phase A ingredients were mixed in a vessel until fully dissolved. In a separate vessel, Phase B ingredients were mixed and heated with low heat until crystals are dissolved. Add Phase B to Phase A, continue mixing until homogeneous.

### Lipstick Formula Procedure

Pre-weigh ingredients of phase A. and the ingredients were mixed and heat to 90-95° C., homogenize until all clear Pulverize phase B and then added to phase A and continue mixing to make sure all in using glass slide. Phase C ingredients were added and continue mix until clear for 5 minutes. Phase D ingredients were added and continue mix until clear for 15 minutes. Check for pigments dispersion using two glass slides. SB-700 was added and mixed well. Pour to mold at 80° C. and refrigerate for 10 minutes.

### Example G: Formulations with B1

### Example G 1

TABLE 1
Nonionic Sunscreen Emulsion

| | | | |
|---|---|---|---|
| | Formula#1% | Formula#2% | Formula#3% |
| Ingredients | W/W | W/W | W/W |

Phase A

| | | | |
|---|---|---|---|
| Water | 60.00 | 60.00 | 60.00 |
| Propylene glycol | 2.00 | 2.00 | 2.00 |
| Carbomer | 0.30 | 0.30 | 0.30 |
| Methylparaben | 1.00 | 1.00 | 1.00 |

Phase B

| | | | |
|---|---|---|---|
| Avobenzone | 3.00 | 3.00 | 3.00 |
| Ethylhexyl Salicylate | 5.00 | 5.00 | 5.00 |
| Octocrylene | 5.00 | 5.00 | 5.00 |
| Benzophenone-3 | 6.00 | 6.00 | 6.00 |
| Homosalate | 10.00 | 10.00 | 10.00 |
| PEG-100 Stearate (and) Glyceryl Stearate | 4.00 | 4.00 | 4.00 |
| C12-15 Alkyl Benzoate | 2.00 | | |
| Benzyl O-toluate | | 2.00 | |
| Phenethyl Benzoate | | | 2.00 |
| Example B1 | 1.00 | 1.00 | 1.00 |

Phase C

| | | | |
|---|---|---|---|
| Sodium hydroxide 10% | 0.70 | 0.70 | 0.70 |
| Total | 100.00 | 100.00 | 100.00 |

### Example G 2

TABLE 2
Water in Silicone mineral (zinc and titanium) emulsion

| | | | |
|---|---|---|---|
| | Formula#1% | Formula#2% | Formula#3% |
| Ingredients | W/W | W/W | W/W |

Phase A

| | | | |
|---|---|---|---|
| Water | 47.50 | 47.50 | 47.50 |
| Glycerin | 5.00 | 5.00 | 5.00 |
| Hydroxyethylcellulose | 0.50 | 0.50 | 0.50 |
| Sodium Chloride | 0.50 | 0.50 | 0.50 |
| Caprylyl Glycol, Phenoxyethanol | 1.50 | 1.50 | 1.50 |

Phase B

| | | | |
|---|---|---|---|
| Zinc Oxide (and) ITT | 8.00 | 8.00 | 8.00 |
| Titanium dioxide (and) ITT | 8.00 | 8.00 | 8.00 |

Phase C

| | | | |
|---|---|---|---|
| Dimethicone(and)Dimethicone/PEG-10/15 | 2.50 | 2.50 | 2.50 |
| Crosspolymer | | | |
| Lauryl PEG-9 Polydimethylsiloxyethyl | 3.00 | 3.00 | 3.00 |
| Dimethicone | | | |
| C12-15 Alkyl Benzoate | 5.00 | — | — |
| Benzyl O-toluate | — | 5.00 | — |
| Phenethyl Benzoate | — | — | 5.00 |
| Phenyl Trimethicone | 4.00 | 4.00 | 4.00 |
| Octyl Palmitate | 5.00 | 5.00 | 5.00 |
| Stearalkonium Hectorite & Caprylic/capric | 2.50 | 2.50 | 2.50 |
| Triglyceride & Propylene Carbonate | | | |
| Example B1 | 2.00 | 2.00 | 2.00 |
| Butyloctyl Salicylate | 5.00 | 5.00 | 5.00 |
| Total | 100 | 100 | 100 |

### Example G 3

TABLE 3
W/Si mineral (zinc) emulsion

| | | | |
|---|---|---|---|
| | Formula#1% | Formula#2% | Formula#3% |
| Ingredients | W/W | W/W | W/W |

Phase A

| | | | |
|---|---|---|---|
| Dimethicone 1cst | 5.00 | 5.00 | 5.00 |
| Dimethicone 2cst | 10.00 | 10.00 | 10.00 |
| Cetyl PEG/PPG-10/1 | 0.50 | 0.50 | 0.50 |
| Dimethicone | | | |
| Polyglyceryl-4 Isostearate (and) | 3.00 | 3.00 | 3.00 |
| Cetyl PEG/PPG-10/1 | | | |
| Dimethicone (and) Hexyl | | | |
| Laurate | | | |
| Caprylic/Capric Triglyceride, | 3.50 | 3.50 | 3.50 |
| Stearalkonium hectorite, | | | |
| Propylene Carbonate | | | |

Phase B

| | | | |
|---|---|---|---|
| Diisopropyl adipate | 1.50 | 1.50 | 1.50 |
| Butyloctyl Salicylate | 5.00 | 5.00 | 5.00 |
| Example B1 | 2.00 | 2.00 | 2.00 |

Phase C

| | | | |
|---|---|---|---|
| Water | 37.82 | 37.82 | 37.82 |
| Hydroxyethyl Cellulose | 0.20 | 0.20 | 0.20 |
| Glycerin | 2.00 | 2.00 | 2.00 |
| C12-15 Alkyl Benzoate | 5.00 | | |
| Benzyl O-toluate | | 5.00 | |
| Phenethyl Benzoate | | | 5.00 |
| Phenoxyethanol (and) Benzoic | 1.00 | 1.00 | 1.00 |
| Acid (and) Dehydroacetic Acid | | | |
| 1,2-Hexanediol | 0.50 | 0.50 | 0.50 |
| Sodium Chloride | 1.00 | 1.00 | 1.00 |

Phase D

| | | | |
|---|---|---|---|
| Zinc Oxide and | 21.98 | 21.98 | 21.98 |
| Triethoxycaprylylsilane | | | |
| Total | 100 | 100 | 100 |

### Example G 4

TABLE 4
Anionic emulsion

| | | | |
|---|---|---|---|
| | Formula#1% | Formula#2% | Formula#3% |
| Ingredients | W/W | W/W | W/W |

Phase A

| | | | |
|---|---|---|---|
| Water | 53.30 | 53.30 | 53.30 |
| Glycerin and Glyceryl Acrylate/Acrylic | 5.00 | 5.00 | 5.00 |
| Acid Copolymer and PVM/MA Copolymer | | | |
| Tetrasodium EDTA | 0.10 | 0.10 | 0.10 |
| Acrylic Acid/VP Crosspolymer | 0.30 | 0.30 | 0.30 |
| Benzoic acid and Dehydroacetic acid in | 1.00 | 1.00 | 1.00 |
| Phenoxyethanol | | | |
| Methylparaben NF | 0.30 | 0.30 | 0.30 |

Phase B

| | | | |
|---|---|---|---|
| Potassium cetyl phosphate | 1.50 | 1.50 | 1.50 |
| Ethylhexyl triazone | 5.00 | 5.00 | 5.00 |
| Avobenzone | 3.00 | 3.00 | 3.00 |
| Bis-ethylhexyloxyphenol Methoxyphenyl | 3.00 | 3.00 | 3.00 |
| Triazine | | | |
| Octisalate | 5.00 | 5.00 | 5.00 |
| Homosalate | 10.00 | 10.00 | 10.00 |
| C12-15 Alkyl Benzoate | 5.00 | | |
| Benzyl O-toluate | | 5.00 | |
| Phenethyl Benzoate | | | 5.00 |
| Dimethicone | 2.00 | 2.00 | 2.00 |
| Behenyl Alcohol and polyglycerol-10 | 1.50 | 1.50 | 1.50 |
| Pentastearate and Sodium Stearoyl Lactate | | | |
| Example B1 | 2.00 | 2.00 | 2.00 |

Phase C

| | | | |
|---|---|---|---|
| NaOH 10% Solution | 2.00 | 2.00 | 2.00 |
| Total | 100.00 | 100.00 | 100.00 |

### Example G 5

TABLE 5
Hydrosheer ™ Spray

| | | | |
|---|---|---|---|
| | Formula#1% | Formula#2% | Formula#3% |
| Ingredients | w/w | w/w | w/w |

Phase A

| | | | |
|---|---|---|---|
| Ethanol | 44.00 | 44.00 | 44.00 |
| Example B1 | 2.00 | 2.00 | 2.00 |

Phase B

| | | | |
|---|---|---|---|
| Avobenzone | 3.00 | 3.00 | 3.00 |
| Octisalate | 5.00 | 5.00 | 5.00 |
| Homosalate | 10.00 | 10.00 | 10.00 |
| Octocrylene | 8.00 | 8.00 | 8.00 |
| Bemotrizinol | 2.00 | 2.00 | 2.00 |
| C12-15 Alkyl Benzoate | 3.00 | | |
| Benzyl O-toluate | | 3.00 | |
| Phenethyl Benzoate | | | 3.00 |
| Diisopropyl Adipate | 5.00 | 5.00 | 5.00 |
| Lauryl Lactate | 5.00 | 5.00 | 5.00 |
| C12-15 Alkyl Lactate | 5.00 | 5.00 | 5.00 |
| Dicaprylyl Carbonate | 4.00 | 4.00 | 4.00 |
| Isodecyl Salicylate | 3.00 | 3.00 | 3.00 |
| Total | 100 | 100 | 100 |

### Example G 6

TABLE 6
Lipstick formula

| | |
|---|---|
| Ingredients | % W/W |

Phase A

| | |
|---|---|
| Ozokerite Wax | 12.97 |
| Polyethylene | 6.25 |
| Octyldodecyl stearate | 6 |
| Diisopropyl adipate | 5 |
| Octyldodecyl Stearoyl Stearate | 5.5 |
| Pentaerythrityl tetraoctanoate | 3.44 |
| C12-15 Alkyl lactate | 4.4 |
| Myristyl lactate | 3.4 |
| Hydrogenated Polyisobutene | 3.7 |
| Example B1 | 2 |
| Octocrylene | 6.52 |
| Ethylhexyl Salicylate | 4.66 |
| Avobenzone | 5.6 |
| Homosalate | 8.36 |

Phase B

| | |
|---|---|
| Caprylyl Glycol (and) Phenoxyethanol | 1.2 |

Phase C

| | |
|---|---|
| Ricinus communis seed oil (and) CI 15850 | 0.2 |
| Ricinus communis seed oil (and) CI 15850 | 0.58 |
| Ricinus communis (Castor) seed oil (and) CI 77891 (and) | 1.54 |
| Polyhydroxystearic Acid | |
| Ricinus communis (Castor)seed oil (and) CI 77491 and C77492 | 0.11 |
| and C177499 and Polyhydroxystearic Acid | |
| Mica (and)Iron Oxides C177491 (and) Titanium Dioxide | 2.99 |
| Synthetic Fluorphogopite and Titanium dioxide and Tin Oxide | 11.17 |
| C177491 and CI 77891 and Synthetic Fluorphlogopite | 3.41 |
| Water (and) Butylene Glycol (and) Hexapeptide-3 | 1 |
| Total | 100 |

### Example H: Formulations with C1

### Example H 1

TABLE 7
Nonionic Sunscreen Emulsion

| | | | |
|---|---|---|---|
| | Formula#1% | Formula#2% | Formula#3% |
| Ingredients | W/W | W/W | W/W |

Phase A

| | | | |
|---|---|---|---|
| Water | 60.00 | 60.00 | 60.00 |
| Propylene glycol | 2.00 | 2.00 | 2.00 |
| Carbomer | 0.30 | 0.30 | 0.30 |
| Methylparaben | 1.00 | 1.00 | 1.00 |

Phase B

| | | | |
|---|---|---|---|
| Avobenzone | 3.00 | 3.00 | 3.00 |
| Ethylhexyl Salicylate | 5.00 | 5.00 | 5.00 |
| Octocrylene | 5.00 | 5.00 | 5.00 |
| Benzophenone-3 | 6.00 | 6.00 | 6.00 |
| Homosalate | 10.00 | 10.00 | 10.00 |
| PEG-100 Stearate (and) Glyceryl Stearate | 4.00 | 4.00 | 4.00 |
| C12-15 Alkyl Benzoate | 2.00 | | |
| Benzyl O-toluate | | 2.00 | |
| Phenethyl Benzoate | | | 2.00 |
| Example C1 | 1.00 | 1.00 | 1.00 |

Phase C

| | | | |
|---|---|---|---|
| Sodium hydroxide 10% | 0.70 | 0.70 | 0.70 |
| Total | 100.00 | 100.00 | 100.00 |

### Example H 2

TABLE 8
Water in Silicone mineral (zinc and titanium) emulsion

| | | | |
|---|---|---|---|
| | Formula#1% | Formula#2% | Formula#3% |
| Ingredients | W/W | W/W | W/W |

Phase A

| | | | |
|---|---|---|---|
| Water | 47.50 | 47.50 | 47.50 |
| Glycerin | 5.00 | 5.00 | 5.00 |
| Hydroxyethylcellulose | 0.50 | 0.50 | 0.50 |
| Sodium Chloride | 0.50 | 0.50 | 0.50 |
| Caprylyl Glycol, Phenoxyethanol | 1.50 | 1.50 | 1.50 |

Phase B

| | | | |
|---|---|---|---|
| Zinc Oxide (and) ITT | 8.00 | 8.00 | 8.00 |
| Titanium dioxide (and) ITT | 8.00 | 8.00 | 8.00 |

Phase C

| | | | |
|---|---|---|---|
| Dimethicone(and)Dimethicone/PEG-10/15 | 2.50 | 2.50 | 2.50 |
| Crosspolymer | | | |
| Lauryl PEG-9 Polydimethylsiloxyethyl | 3.00 | 3.00 | 3.00 |
| Dimethicone | | | |
| C12-15 Alkyl Benzoate | 5.00 | — | — |
| Benzyl O-toluate | — | 5.00 | — |
| Phenethyl Benzoate | — | — | 5.00 |
| Phenyl Trimethicone | 4.00 | 4.00 | 4.00 |
| Octyl Palmitate | 5.00 | 5.00 | 5.00 |
| Stearalkonium Hectorite & Caprylic/capric | 2.50 | 2.50 | 2.50 |
| Triglyceride & Propylene Carbonate | | | |
| Example C1 | 2.00 | 2.00 | 2.00 |
| Butyloctyl Salicylate | 5.00 | 5.00 | 5.00 |
| Total | 100 | 100 | 100 |

### Example H 3

TABLE 9
W/Si mineral (zinc) emulsion

| | | | |
|---|---|---|---|
| | Formula#1% | Formula#2% | Formula#3% |
| Ingredients | W/W | W/W | W/W |

Phase A

| | | | |
|---|---|---|---|
| Dimethicone 1cst | 5.00 | 5.00 | 5.00 |
| Dimethicone 2cst | 10.00 | 10.00 | 10.00 |
| Cetyl PEG/PPG-10/1 Dimethicone | 0.50 | 0.50 | 0.50 |
| Polyglyceryl-4 Isostearate (and) Cetyl PEG/PPG- | 3.00 | 3.00 | 3.00 |
| 10/1 Dimethicone (and) Hexyl Laurate | | | |
| Caprylic/Capric Triglyceride, Stearalkonium | 3.50 | 3.50 | 3.50 |
| hectorite, Propylene Carbonate | | | |

Phase B

| | | | |
|---|---|---|---|
| Diisopropyl adipate | 1.50 | 1.50 | 1.50 |
| Butyloctyl Salicylate | 5.00 | 5.00 | 5.00 |
| Example C1 | 2.00 | 2.00 | 2.00 |

Phase C

| | | | |
|---|---|---|---|
| Water | 37.82 | 37.82 | 37.82 |
| Hydroxyethyl Cellulose | 0.20 | 0.20 | 0.20 |
| Glycerin | 2.00 | 2.00 | 2.00 |
| C12-15 Alkyl Benzoate | 5.00 | | |
| Benzyl O-toluate | | 5.00 | |
| Phenethyl Benzoate | | | 5.00 |
| Phenoxyethanol (and) Benzoic Acid (and) | 1.00 | 1.00 | 1.00 |
| Dehydroacetic Acid | | | |
| 1,2-Hexanediol | 0.50 | 0.50 | 0.50 |
| Sodium Chloride | 1.00 | 1.00 | 1.00 |

Phase D

| | | | |
|---|---|---|---|
| Zinc Oxide and Triethoxycaprylylsilane | 21.98 | 21.98 | 21.98 |
| Total | 100 | 100 | 100 |

### Example H 4

TABLE 10
Anionic emulsion

| | | | |
|---|---|---|---|
| | Formula | Formula | Formula# |
| | #1 % | #2 % | 3 % |
| Ingredients | W/W | W/W | W/W |

Phase A

| | | | |
|---|---|---|---|
| Water | 53.30 | 53.30 | 53.30 |
| Glycerin and Glyceryl Acrylate/Acrylic | 5.00 | 5.00 | 5.00 |
| Acid Copolymer and PVM/MA Copolymer | | | |
| Tetrasodium EDTA | 0.10 | 0.10 | 0.10 |
| Acrylic Acid/VP Crosspolymer | 0.30 | 0.30 | 0.30 |
| OPTIPHEN ™ ND | 1.00 | 1.00 | 1.00 |
| Methylparaben NF | 0.30 | 0.30 | 0.30 |

Phase B

| | | | |
|---|---|---|---|
| Potassium cetyl phosphate | 1.50 | 1.50 | 1.50 |
| Univol T 150 | 5.00 | 5.00 | 5.00 |
| Escalol 517 (Avobenzone) | 3.00 | 3.00 | 3.00 |
| Bis-ethylhexyloxyphenol Methoxyphenyl | 3.00 | 3.00 | 3.00 |
| Triazine | | | |
| Escalol 587 (Octisalate) | 5.00 | 5.00 | 5.00 |
| Homosalate | 10.00 | 10.00 | 10.00 |
| C12-15 Alkyl Benzoate | 5.00 | | |
| Benzyl O-toluate | | 5.00 | |
| Phenethyl Benzoate | | | 5.00 |
| Dimethicone | 2.00 | 2.00 | 2.00 |
| Behenyl Alcohol and polyglycerol-10 | 1.50 | 1.50 | 1.50 |
| Pentastearate and Sodium Stearoyl Lactate | | | |
| Example C1 | 2.00 | 2.00 | 2.00 |

Phase C

| | | | |
|---|---|---|---|
| NaOH 10% Solution | 2.00 | 2.00 | 2.00 |
| Total | 100.00 | 100.00 | 100.00 |

### Example H 5

TABLE 11
Hydrosheer ™ Spray

| | | | |
|---|---|---|---|
| | Formula#1 | Formula#2 | Formula#3 |
| Ingredient | % w/w | % w/w | % w/w |

Phase A

| | | | |
|---|---|---|---|
| Ethanol | 44.00 | 44.00 | 44.00 |
| Example C1 | 2.00 | 2.00 | 2.00 |

Phase B

| | | | |
|---|---|---|---|
| Avobenzone | 3.00 | 3.00 | 3.00 |
| Octisalate | 5.00 | 5.00 | 5.00 |
| Homosalate | 10.00 | 10.00 | 10.00 |
| Octocrylene | 8.00 | 8.00 | 8.00 |
| Bemotrizinol | 2.00 | 2.00 | 2.00 |
| C12-15 Alkyl Benzoate | 3.00 | | |
| Benzyl O-toluate | | 3.00 | |
| Phenethyl Benzoate | | | 3.00 |
| Diisopropyl Adipate | 5.00 | 5.00 | 5.00 |
| Lauryl Lactate | 5.00 | 5.00 | 5.00 |
| C12-15 Alkyl Lactate | 5.00 | 5.00 | 5.00 |
| Dicaprylyl Carbonate | 4.00 | 4.00 | 4.00 |
| Isodecyl Salicylate | 3.00 | 3.00 | 3.00 |
| Total | 100 | 100 | 100 |

### Example H 6

TABLE 12
Lipstick formula

| | |
|---|---|
| Ingredients | % W/W |

Phase A

| | |
|---|---|
| Ozokerite Wax | 12.97 |
| Polyethylene | 6.25 |
| Octyldodecyl stearate | 6 |
| Diisopropyl adipate | 5 |
| Octyldodecyl Stearoyl Stearate | 5.5 |
| Pentaerythrityl tetraoctanoate | 3.44 |
| C12-15 Alkyl lactate | 4.4 |
| Myristyl lactate | 3.4 |
| Hydrogenated Polyisobutene | 3.7 |
| Example C1 | 2 |
| Octocrylene | 6.52 |
| Ethylhexyl Salicylate | 4.66 |
| Avobenzone | 5.6 |
| Homosalate | 8.36 |

Phase B

| | |
|---|---|
| Caprylyl Glycol (and) Phenoxyethanol | 1.2 |

Phase C

| | |
|---|---|
| Ricinus communis seed oil (and) CI 15850 | 0.2 |
| Ricinus communis seed oil (and) CI 15850 | 0.58 |
| Ricinus communis (Castor) seed oil (and) CI 77891 (and) | 1.54 |
| Polyhydroxystearic Acid | |
| Ricinus communis (Castor)seed oil (and) CI 77491 and C77492 | 0.11 |
| and C177499 and Polyhydroxystearic Acid | |
| Mica (and)Iron Oxides C177491 (and) Titanium Dioxide | 2.99 |
| Synthetic Fluorphogopite and Titanium dioxide and Tin Oxide | 11.17 |
| C177491 and CI 77891 and Synthetic Fluorphlogopite | 3.41 |
| Water (and) Butylene Glycol (and) Hexapeptide-3 | 1 |
| Total | 100 |

### Example I: Formulations with C4

### Example I1

TABLE 13
Nonionic Sunscreen Emulsion

| | | | |
|---|---|---|---|
| | For- | For- | For- |
| | mula#1 % | mula#2 % | mula#3 % |
| Ingredients | W/W | W/W | W/W |

Phase A

| | | | |
|---|---|---|---|
| Water | 60.00 | 60.00 | 60.00 |
| Propylene glycol | 2.00 | 2.00 | 2.00 |
| Carbomer | 0.30 | 0.30 | 0.30 |
| Methylparaben | 1.00 | 1.00 | 1.00 |

Phase B

| | | | |
|---|---|---|---|
| Avobenzone | 3.00 | 3.00 | 3.00 |
| Ethylhexyl Salicylate | 5.00 | 5.00 | 5.00 |
| Octocrylene | 5.00 | 5.00 | 5.00 |
| Benzophenone-3 | 6.00 | 6.00 | 6.00 |
| Homosalate | 10.00 | 10.00 | 10.00 |
| PEG-100 Stearate (and) Glyceryl | 4.00 | 4.00 | 4.00 |
| Stearate | | | |
| C12-15 Alkyl Benzoate | 2.00 | | |
| Benzyl O-toluate | | 2.00 | |
| Phenethyl Benzoate | | | 2.00 |
| Example C4 | 1.00 | 1.00 | 1.00 |

Phase C

| | | | |
|---|---|---|---|
| Sodium hydroxide 10% | 0.70 | 0.70 | 0.70 |
| Total | 100.00 | 100.00 | 100.00 |

### Example I 2

TABLE 14
Water in Silicone mineral (zinc and titanium) emulsion

| | | | |
|---|---|---|---|
| | Formu- | Formu- | Formu- |
| | la#1 % | la#2 % | la#3 % |
| Ingredients | W/W | W/W | W/W |

Phase A

| | | | |
|---|---|---|---|
| Water | 47.50 | 47.50 | 47.50 |
| Glycerin | 5.00 | 5.00 | 5.00 |
| Hydroxyethylcellulose | 0.50 | 0.50 | 0.50 |
| Sodium Chloride | 0.50 | 0.50 | 0.50 |
| Caprylyl Glycol, Phenoxyethanol | 1.50 | 1.50 | 1.50 |

Phase B

| | | | |
|---|---|---|---|
| Zinc Oxide (and) ITT | 8.00 | 8.00 | 8.00 |
| Titanium dioxide (and) ITT | 8.00 | 8.00 | 8.00 |

Phase C

| | | | |
|---|---|---|---|
| Dimethicone(and)Dimethicone/PEG-10/15 | 2.50 | 2.50 | 2.50 |
| Crosspolymer | | | |
| Lauryl PEG-9 Polydimethylsiloxyethyl | 3.00 | 3.00 | 3.00 |
| Dimethicone | | | |
| C12-15 Alkyl Benzoate | 5.00 | — | — |
| Benzyl O-toluate | — | 5.00 | — |
| Phenethyl Benzoate | — | — | 5.00 |
| Phenyl Trimethicone | 4.00 | 4.00 | 4.00 |
| Octyl Palmitate | 5.00 | 5.00 | 5.00 |
| Stearalkonium Hectorite & Caprylic/capric | 2.50 | 2.50 | 2.50 |
| Triglyceride & Propylene Carbonate | | | |
| Example C4 | 2.00 | 2.00 | 2.00 |
| Butyloctyl Salicylate | 5.00 | 5.00 | 5.00 |
| Total | 100 | 100 | 100 |

### Example I 3

TABLE 15
W/Si mineral (zinc) emulsion

| | | | |
|---|---|---|---|
| | Formu- | Formu- | Formu- |
| | la#1 % | la#2 % | la#3 % |
| Ingredients | W/W | W/W | W/W |

Phase A

| | | | |
|---|---|---|---|
| Dimethicone 1cst | 5.00 | 5.00 | 5.00 |
| Dimethicone 2cst | 10.00 | 10.00 | 10.00 |
| Cetyl PEG/PPG -10/1 Dimethicone | 0.50 | 0.50 | 0.50 |
| Polyglyceryl-4 Isostearate (and) Cetyl | | | |
| PEG/PPG-10/1 Dimethicone (and) Hexyl | 3.00 | 3.00 | 3.00 |
| Laurate | | | |
| Caprylic/Capric Triglyceride, | 3.50 | 3.50 | 3.50 |
| Stearalkonium hectorite, Propylene | | | |
| Carbonate | | | |

Phase B

| | | | |
|---|---|---|---|
| Diisopropyl adipate | 1.50 | 1.50 | 1.50 |
| Butyloctyl Salicylate | 5.00 | 5.00 | 5.00 |
| Example C4 | 2.00 | 2.00 | 2.00 |

Phase C

| | | | |
|---|---|---|---|
| Water | 37.82 | 37.82 | 37.82 |
| Hydroxyethyl Cellulose | 0.20 | 0.20 | 0.20 |
| Glycerin | 2.00 | 2.00 | 2.00 |
| C12-15 Alkyl Benzoate | 5.00 | | |
| Benzyl O-toluate | | 5.00 | |
| Phenethyl Benzoate | | | 5.00 |
| Phenoxyethanol (and) Benzoic Acid (and) | 1.00 | 1.00 | 1.00 |
| Dehydroacetic Acid | | | |
| 1,2-Hexanediol | 0.50 | 0.50 | 0.50 |
| Sodium Chloride | 1.00 | 1.00 | 1.00 |
| Phase D | | | |
| Zinc Oxide and Triethoxycaprylylsilane | 21.98 | 21.98 | 21.98 |
| Total | 100 | 100 | 100 |

### Example I 4

TABLE 16
Anionic emulsion

| | | | |
|---|---|---|---|
| | Formu- | Formu- | Formu- |
| | la#1 % | la#2 % | la#3 % |
| Ingredients | W/W | W/W | W/W |

Phase A

| | | | |
|---|---|---|---|
| Water | 53.30 | 53.30 | 53.30 |
| Glycerin and Glyceryl Acrylate/Acrylic | 5.00 | 5.00 | 5.00 |
| Acid Copolymer and PVM/MA Copolymer | | | |
| Tetrasodium EDTA | 0.10 | 0.10 | 0.10 |
| Acrylic Acid/VP Crosspolymer | 0.30 | 0.30 | 0.30 |
| OPTIPHEN ™ ND | 1.00 | 1.00 | 1.00 |
| Methylparaben NF | 0.30 | 0.30 | 0.30 |

Phase B

| | | | |
|---|---|---|---|
| Potassium cetyl phosphate | 1.50 | 1.50 | 1.50 |
| Univol T 150 | 5.00 | 5.00 | 5.00 |
| Avobenzone | 3.00 | 3.00 | 3.00 |
| Bis-ethylhexyloxyphenol Methoxyphenyl | 3.00 | 3.00 | 3.00 |
| Triazine | | | |
| Octisalate | 5.00 | 5.00 | 5.00 |
| Homosalate | 10.00 | 10.00 | 10.00 |
| C12-15 Alkyl Benzoate | 5.00 | | |
| Benzyl O-toluate | | 5.00 | |
| Phenethyl Benzoate | | | 5.00 |
| Dimethicone | 2.00 | 2.00 | 2.00 |
| Behenyl Alcohol and polyglycerol-10 | 1.50 | 1.50 | 1.50 |
| Pentastearate and Sodium Stearoyl Lactate | | | |
| Example C4 | 2.00 | 2.00 | 2.00 |

Phase C

| | | | |
|---|---|---|---|
| NaOH 10% Solution | 2.00 | 2.00 | 2.00 |
| Total | 100.00 | 100.00 | 100.00 |

### Example I5

TABLE 17
Hydrosheer ™ Spray

| | | | |
|---|---|---|---|
| | Formu- | Formu- | Formu- |
| | la#1 % | la#2 % | la#3 % |
| Ingredient | W/W | W/W | W/W |

Phase A

| | | | |
|---|---|---|---|
| Ethanol | 44.00 | 44.00 | 44.00 |
| Example C4 | 2.00 | 2.00 | 2.00 |

Phase B

| | | | |
|---|---|---|---|
| Avobenzone | 3.00 | 3.00 | 3.00 |
| Octisalate | 5.00 | 5.00 | 5.00 |
| Homosalate | 10.00 | 10.00 | 10.00 |
| Octocrylene | 8.00 | 8.00 | 8.00 |
| Bemotrizinol | 2.00 | 2.00 | 2.00 |
| C12-15 Alkyl Benzoate | 3.00 | | |
| Benzyl O-toluate | | 3.00 | |
| Phenethyl Benzoate | | | 3.00 |
| Diisopropyl Adipate | 5.00 | 5.00 | 5.00 |
| Lauryl Lactate | 5.00 | 5.00 | 5.00 |
| C12-15 Alkyl Lactate | 5.00 | 5.00 | 5.00 |
| Dicaprylyl Carbonate | 4.00 | 4.00 | 4.00 |
| Isodecyl Salicylate | 3.00 | 3.00 | 3.00 |
| Total | 100 | 100 | 100 |

### Example I 6

TABLE 18
Lipstick formula

| | |
|---|---|
| Ingredients | % W/W |

Phase A

| | |
|---|---|
| Ozokerite Wax | 12.97 |
| Polyethylene | 6.25 |
| Octyldodecyl stearate | 6 |
| Diisopropyl adipate | 5 |
| Octyldodecyl Stearoyl Stearate | 5.5 |
| Pentaerythrityl tetraoctanoate | 3.44 |
| C12-15 Alkyl lactate | 4.4 |
| Myristyl lactate | 3.4 |
| Hydrogenated Polyisobutene | 3.7 |
| Example C4 | 2 |
| Octocrylene | 6.52 |
| Ethylhexyl Salicylate | 4.66 |
| Avobenzone | 5.6 |
| Homosalate | 8.36 |

Phase B

| | |
|---|---|
| Caprylyl Glycol (and) Phenoxyethanol | 1.2 |

Phase C

| | |
|---|---|
| Ricinus communis seed oil (and) CI 15850 | 0.2 |
| Ricinus communis seed oil (and) CI 15850 | 0.58 |
| Ricinus communis (Castor) seed oil (and) | 1.54 |
| CI 77891 (and) Polyhydroxystearic Acid | |
| Ricinus communis (Castor)seed oil (and) | 0.11 |
| CI 77491 and C77492 and C177499 and Polyhydroxystearic Acid | |
| Mica (and)Iron Oxides C177491 (and) Titanium Dioxide | 2.99 |
| Synthetic Fluorphogopite and Titanium dioxide and Tin Oxide | 11.17 |
| C177491 and CI 77891 and Synthetic Fluorphlogopite | 3.41 |
| Water (and) Butylene Glycol (and) Hexapeptide-3 | 1 |
| Total | 100 |

### Example J: Formulations with D1 or D2

### Example J 1

TABLE 19
Nonionic Sunscreen Emulsion

| | | | |
|---|---|---|---|
| | Formu- | Formu- | Formu- |
| | la 1 % | la 2 % | la 3 % |
| Ingredients | W/W | W/W | W/W |

Phase A

| | | | |
|---|---|---|---|
| Water | 60.00 | 60.00 | 60.00 |
| Propylene glycol | 2.00 | 2.00 | 2.00 |
| Carbomer | 0.30 | 0.30 | 0.30 |
| Methylparaben | 1.00 | 1.00 | 1.00 |

Phase B

| | | | |
|---|---|---|---|
| Avobenzone | 3.00 | 3.00 | 3.00 |
| Ethylhexyl Salicylate | 5.00 | 5.00 | 5.00 |
| Octocrylene | 5.00 | 5.00 | 5.00 |
| Benzophenone-3 | 6.00 | 6.00 | 6.00 |
| Homosalate | 10.00 | 10.00 | 10.00 |
| PEG-100 Stearate (and) Glyceryl Stearate | 4.00 | 4.00 | 4.00 |
| C12-15 Alkyl Benzoate | 2.00 | | |
| Benzyl O-toluate | | 2.00 | |
| Phenethyl Benzoate | | | 2.00 |
| Example D1 or D2 | 1.00 | 1.00 | 1.00 |

Phase C

| | | | |
|---|---|---|---|
| Sodium hydroxide 10% | 0.70 | 0.70 | 0.70 |
| Total | 100.00 | 100.00 | 100.00 |

### Example J 2

TABLE 20
Water in Silicone mineral (zinc and titanium) emulsion

| | | | |
|---|---|---|---|
| | Formu- | Formu- | Formu- |
| | la#1 % | la#2 % | la#3 % |
| Ingredients | W/W | W/W | W/W |

Phase A

| | | | |
|---|---|---|---|
| Water | 47.50 | 47.50 | 47.50 |
| Glycerin | 5.00 | 5.00 | 5.00 |
| Hydroxyethylcellulose | 0.50 | 0.50 | 0.50 |
| Sodium Chloride | 0.50 | 0.50 | 0.50 |
| Caprylyl Glycol, Phenoxyethanol | 1.50 | 1.50 | 1.50 |

Phase B

| | | | |
|---|---|---|---|
| Zinc Oxide (and) ITT | 8.00 | 8.00 | 8.00 |
| Titanium dioxide (and) ITT | 8.00 | 8.00 | 8.00 |

Phase C

| | | | |
|---|---|---|---|
| Dimethicone(and)Dimethicone/PEG- | 2.50 | 2.50 | 2.50 |
| 10/15 Crosspolymer | | | |
| Lauryl PEG-9 Polydimethylsiloxyethyl | 3.00 | 3.00 | 3.00 |
| Dimethicone | | | |
| C12-15 Alkyl Benzoate | 5.00 | — | — |
| Benzyl O-toluate | — | 5.00 | — |
| Phenethyl Benzoate | — | — | 5.00 |
| Phenyl Trimethicone | 4.00 | 4.00 | 4.00 |
| Octyl Palmitate | 5.00 | 5.00 | 5.00 |
| Stearalkonium Hectorite & | 2.50 | 2.50 | 2.50 |
| Caprylic/capric Triglyceride & Propylene | | | |
| Carbonate | | | |
| Example D1 or D2 | 2.00 | 2.00 | 2.00 |
| Butyloctyl Salicylate | 5.00 | 5.00 | 5.00 |
| Total | 100 | 100 | 100 |

### Example J 3

TABLE 21
W/Si mineral (zinc) emulsion

| | | | |
|---|---|---|---|
| | For- | For- | For- |
| | mula#1 | mula#2 | mula#3 |
| Ingredients | % W/W | % W/W | % W/W |

Phase A

| | | | |
|---|---|---|---|
| Dimethicone 1cst | 5.00 | 5.00 | 5.00 |
| Dimethicone 2cst | 10.00 | 10.00 | 10.00 |
| Cetyl PEG/PPG -10/1 Dimethicone | 0.50 | 0.50 | 0.50 |
| Polyglyceryl-4 Isostearate (and) | 3.00 | 3.00 | 3.00 |
| Cetyl PEG/PPG-10/1 | | | |
| Dimethicone (and) Hexyl Laurate | | | |
| Caprylic/Capric Triglyceride, | 3.50 | 3.50 | 3.50 |
| Stearalkonium hectorite, | | | |
| Propylene Carbonate | | | |

Phase B

| | | | |
|---|---|---|---|
| Diisopropyl adipate | 1.50 | 1.50 | 1.50 |
| Butyloctyl Salicylate | 5.00 | 5.00 | 5.00 |
| Example D1 or D2 | 2.00 | 2.00 | 2.00 |

Phase C

| | | | |
|---|---|---|---|
| Water | 37.82 | 37.82 | 37.82 |
| Hydroxyethyl Cellulose | 0.20 | 0.20 | 0.20 |
| Glycerin | 2.00 | 2.00 | 2.00 |
| C12-15 Alkyl Benzoate | 5.00 | | |
| Benzyl O-toluate | | 5.00 | |
| Phenethyl Benzoate | | | 5.00 |
| Phenoxyethanol (and) Benzoic | 1.00 | 1.00 | 1.00 |
| Acid (and) Dehydroacetic Acid | | | |
| 1,2-Hexanediol | 0.50 | 0.50 | 0.50 |
| Sodium Chloride | 1.00 | 1.00 | 1.00 |
| Phase D | | | |
| Zinc Oxide and Triethoxycaprylylsilane | 21.98 | 21.98 | 21.98 |
| Total | 100 | 100 | 100 |

### Example J 4

TABLE 22
Anionic emulsion

| | | | |
|---|---|---|---|
| | Formula#1 | Formula#2 | Formula#3 |
| Ingredients | % W/W | % W/W | % W/W |

Phase A

| | | | |
|---|---|---|---|
| Water | 53.30 | 53.30 | 53.30 |
| Glycerin and Glyceryl | 5.00 | 5.00 | 5.00 |
| Acrylate/Acrylic Acid | | | |
| Copolymer and | | | |
| PVM/MA Copolymer | | | |
| Tetrasodium EDTA | 0.10 | 0.10 | 0.10 |
| Acrylic Acid/VP Crosspolymer | 0.30 | 0.30 | 0.30 |
| OPTIPHEN ™ ND | 1.00 | 1.00 | 1.00 |
| Methylparaben NF | 0.30 | 0.30 | 0.30 |

Phase B

| | | | |
|---|---|---|---|
| Potassium cetyl phosphate | 1.50 | 1.50 | 1.50 |
| Univol T 150 | 5.00 | 5.00 | 5.00 |
| Avobenzone | 3.00 | 3.00 | 3.00 |
| Bis-ethylhexyloxyphenol | 3.00 | 3.00 | 3.00 |
| Methoxyphenyl Triazine | | | |
| Octisalate | 5.00 | 5.00 | 5.00 |
| Homosalate | 10.00 | 10.00 | 10.00 |
| C12-15 Alkyl Benzoate | 5.00 | | |
| Benzyl O-toluate | | 5.00 | |
| Phenethyl Benzoate | | | 5.00 |
| Dimethicone | 2.00 | 2.00 | 2.00 |
| Behenyl Alcohol and | 1.50 | 1.50 | 1.50 |
| polyglycerol-10 Pentastearate | | | |
| and Sodium Stearoyl Lactate | | | |
| Example D1 or D2 | 2.00 | 2.00 | 2.00 |
| Phase C | | | |
| NaOH 10% Solution | 2.00 | 2.00 | 2.00 |
| Total | 100.00 | 100.00 | 100.00 |

### Example J 5

TABLE 23
Hydrosheer ™ Spray

| | | | |
|---|---|---|---|
| | Formula#1 | Formula#2 | Formula#3 |
| Ingredients | % W/W | % W/W | % W/W |

Phase A

| | | | |
|---|---|---|---|
| Ethanol | 44.00 | 44.00 | 44.00 |
| Example D1 or D2 | 2.00 | 2.00 | 2.00 |

Phase B

| | | | |
|---|---|---|---|
| Avobenzone | 3.00 | 3.00 | 3.00 |
| Octisalate | 5.00 | 5.00 | 5.00 |
| Homosalate | 10.00 | 10.00 | 10.00 |
| Octocrylene | 8.00 | 8.00 | 8.00 |
| Bemotrizinol | 2.00 | 2.00 | 2.00 |
| C12-15 Alkyl Benzoate | 3.00 | | |
| Benzyl O-toluate | | 3.00 | |
| Phenethyl Benzoate | | | 3.00 |
| Diisopropyl Adipate | 5.00 | 5.00 | 5.00 |
| Lauryl Lactate | 5.00 | 5.00 | 5.00 |
| C12-15 Alkyl Lactate | 5.00 | 5.00 | 5.00 |
| Dicaprylyl Carbonate | 4.00 | 4.00 | 4.00 |
| Isodecyl Salicylate | 3.00 | 3.00 | 3.00 |
| Total | 100 | 100 | 100 |

### Example J 6

TABLE 24
Lipstick formula

| | |
|---|---|
| Ingredients | % W/W |

Phase A

| | |
|---|---|
| Ozokerite Wax | 12.97 |
| Polyethylene | 6.25 |
| Octyldodecyl stearate | 6 |
| Diisopropyl adipate | 5 |
| Octyldodecyl Stearoyl Stearate | 5.5 |
| Pentaerythrityl tetraoctanoate | 3.44 |
| C12-15 Alkyl lactate | 4.4 |
| Myristyl lactate | 3.4 |
| Hydrogenated Polyisobutene | 3.7 |
| Example D1 or D2 | 2 |
| Octocrylene | 6.52 |
| Ethylhexyl Salicylate | 4.66 |
| Avobenzone | 5.6 |
| Homosalate | 8.36 |

Phase B

| | |
|---|---|
| Caprylyl Glycol (and) Phenoxyethanol | 1.2 |

Phase C

| | |
|---|---|
| Ricinus communis seed oil (and) | 0.2 |
| CI [symbol] 150 | |
| Ricinus communis seed oil (and) | 0.58 |
| CI 15850 | |
| Ricinus communis (Castor) seed | 1.54 |
| oil (and) CI 77891 (and) | |
| Polyhydroxystearic Acid | |
| Ricinus communis (Castor)seed oil | 0.11 |
| (and) CI 77491 and [symbol] 7492 | |
| and C177499 and Polyhydroxystearic | |
| Acid | |
| Mica (and)Iron Oxides C177491 (and) | 2.99 |
| Titanium Dioxide | |
| Synthetic Fluorphogopite and | 11.17 |
| Titanium dioxide and Tin Oxide | |
| C177491 and CI 77891 and Synthetic | 3.41 |
| Fluorphlogopite | |
| Water (and) Butylene Glycol (and) | 1 |
| Hexapeptide-3 | |
| Total | 100 |

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed and/or claimed inventive concept(s).

We claim:

1. A composition comprising a reaction product of
(a) a maleated natural oil, comprising a natural oil with maleated functionality; and
(b) a functionalized or unfunctionalized moiety selected from the group consisting of hydrophobic moieties, hydrophilic moieties, and combinations thereof,
with the proviso that the hydrophilic moiety is not a glycerol moiety.

2. The composition according to claim 1, wherein the reaction product comprises an unreacted maleated functionality.

3. The composition according to claim 1, wherein the reaction product comprises a maleated functionality functionalized with a hydrophobic moiety, a hydrophilic moiety, or combinations thereof.

4. The composition according to claim 1, wherein the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof.

5. The composition according to claim 1, wherein the hydrophilic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₁ to about C₅ atoms, unsubstituted or substituted alkyl, cycloalkyl, alkenyl, and aryl amines, wherein any of the beforehand mentioned groups may be with or without heteroatoms, containing from about C₁ to about C₅ atoms, unsubstituted or substituted polyols, wherein any of the beforehand mentioned groups may be with or without heteroatoms, containing from about C₂ to about C₃₆ atoms, silanes, and combinations thereof.

6. The composition according to claim 5, wherein the silane is functionalized with an alcohol or an amine, and combinations thereof.

7. The composition according to claim 4, wherein the hydrophobic alcohol is selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof.

8. The composition according to claim 5, wherein the hydrophilic alcohol is selected from the group consisting of methanol, ethanol, propanol, isopropanol, butanol, ethylene glycol, propylene glycol, diethylene glycol, dipropylene glycol, dibutylene glycol, polyethylene glycol, methoxypolyethylene glycol, polypropylene glycol, hexylene glycol, sorbitol, neopentylglycol, eythritol, mannitol, xylitol, threitol, pentaerythritol, beta-cyclodextrin, L-Ribose, 2-deoxy-D-galactose, and mixtures thereof.

9. The composition according to claim 4, wherein the hydrophobic amine is selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof.

10. The composition according to claim 5, wherein the hydrophilic amine is selected from the group consisting of 2-methylpentane-1,5-diamine, diethanol amine, diisopropanolamine, serinol hydrochloride, 2-amino-2-ethyl-1,3-propanediol, N-methyl-D-glucosamine, D-galactosamine hydrochloride, D-glucosamine hydrochloride, D-mannosamine hydrochloride, and mixtures thereof.

11. The composition according to claim 4, wherein the silicon-based compound is a hydrophobic compound selected from the group consisting of aminopropylmethylsiloxane-dimethylsiloxane, N-ethylaminoisobutyl terminated polydimethylsiloxane, poly(1,1-dimethyl silazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

12. The composition according to claim 5, wherein the silane is a hydrophilic compound selected from the group consisting of 3-aminopropylsilanetriol, N-(2-aminoethyl)-3-aminopropylsilanetriol, and mixtures thereof.

13. The composition according to claim 1, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof.

14. The composition according to claim 13, wherein the maleated natural oil is a maleated soybean oil.

15. The composition according to claim 1, wherein the composition is selected from the group consisting of skin care compositions, oral care compositions, hair care compositions, energy compositions, construction compositions, biocidal compositions, preservative compositions, nutraceutical compositions, food compositions, agricultural compositions, coating compositions, cosmetic compositions, homecare compositions, industrial and institutional compositions, textile compositions, laundry compositions, cleaning compositions, and disinfecting compositions.

16. A composition comprising a reaction product of
(a) a maleated natural oil, comprising a natural oil with maleated functionality;
(b) a functionalized or unfunctionalized hydrophobic moiety; and
(c) a glycerol moiety.

17. The composition according to claim 16, wherein the reaction product comprises an unreacted maleated functionality.

18. The composition according to claim 16, wherein the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof.

19. The composition according to claim 18 wherein the hydrophobic moiety is an alcohol selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof.

20. The composition according to claim 18, wherein the hydrophobic moiety is an amine selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof.

21. The composition according to claim 18, wherein the silicon-based compound is a hydrophobic compound selected from the group consisting of aminopropylmethylsiloxane-dimethylsiloxane, N-ethylaminoisobutyl terminated polydimethylsiloxane, poly(1,1-dimethyl silazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

22. The composition according to claim 16, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof.

23. The composition according to claim 22, wherein the maleated natural oil is a maleated soybean oil.

24. The composition according to claim 16, wherein the composition is selected from the group consisting of skin care compositions, oral care compositions, hair care compositions, energy compositions, construction compositions, biocidal compositions, preservative compositions, nutraceutical compositions, food compositions, agricultural compositions, coating compositions, cosmetic compositions, homecare compositions, industrial and institutional compositions, textile compositions, laundry compositions, cleaning compositions, and disinfecting compositions.

25. The composition according to claim 16, wherein the compound is selected from the group of structures represented by the structures set out below:

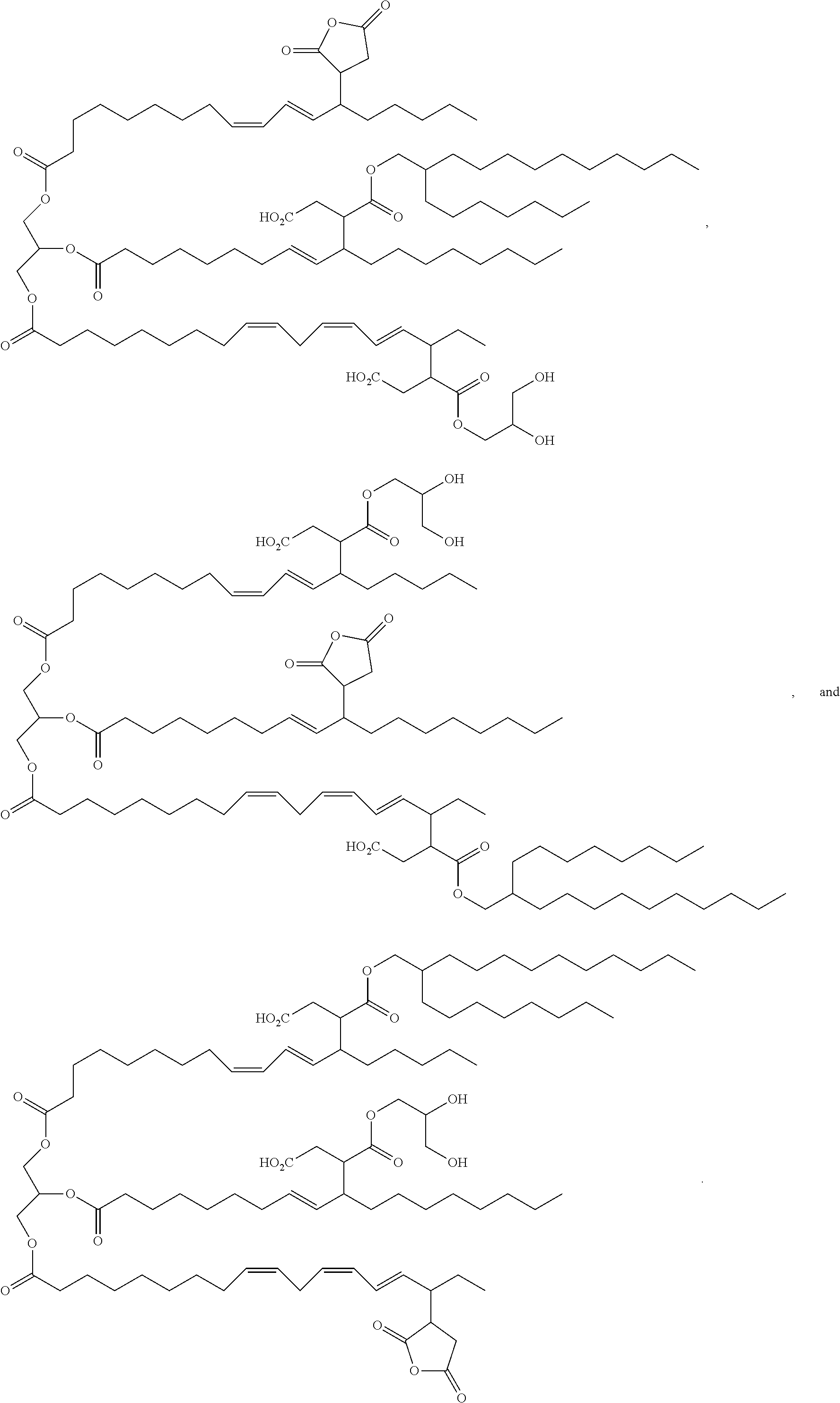

26. A compound selected from the group of structures represented by the structures set out below:

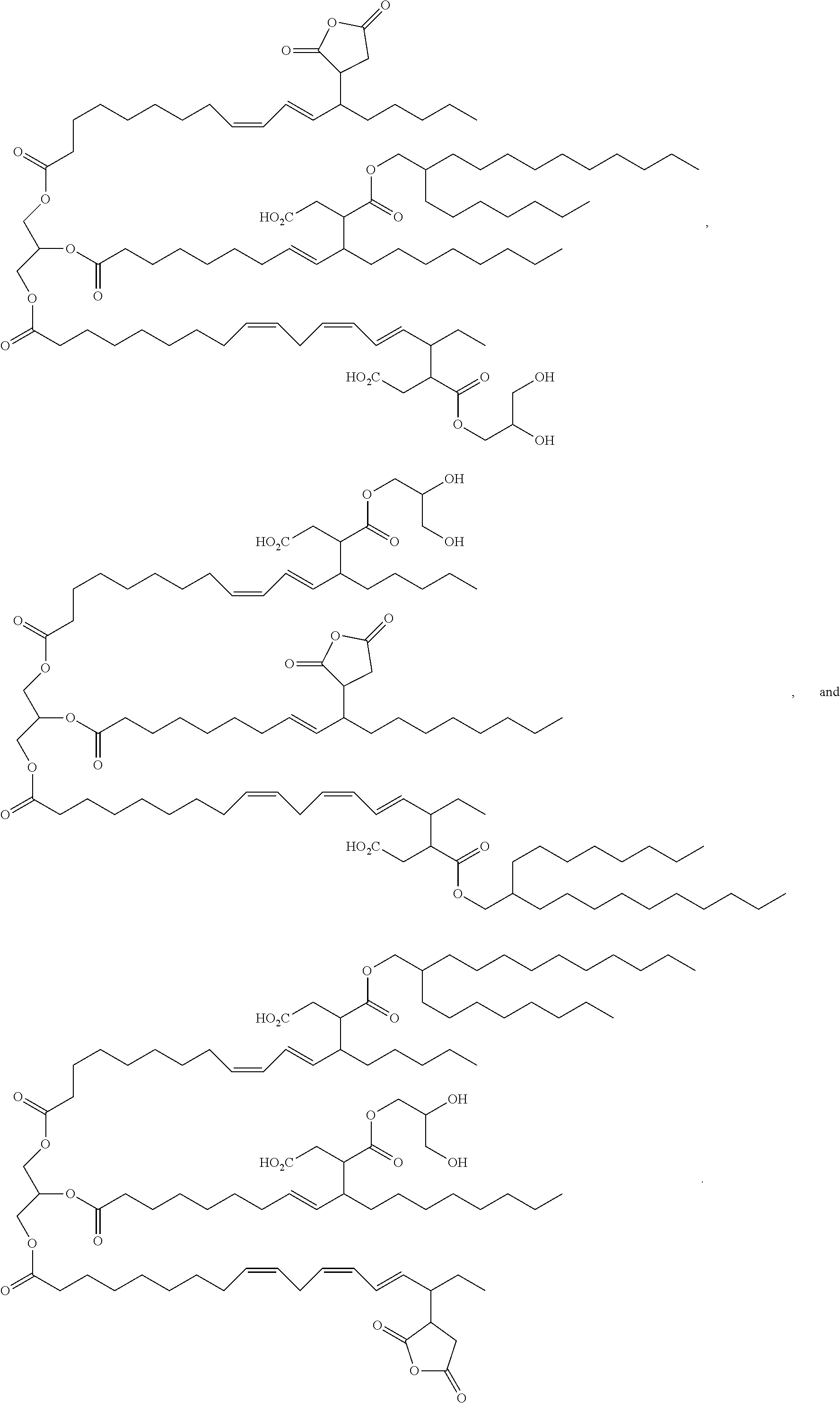

27. A composition comprising a compound selected from the group of structures represented by the structures set out below:

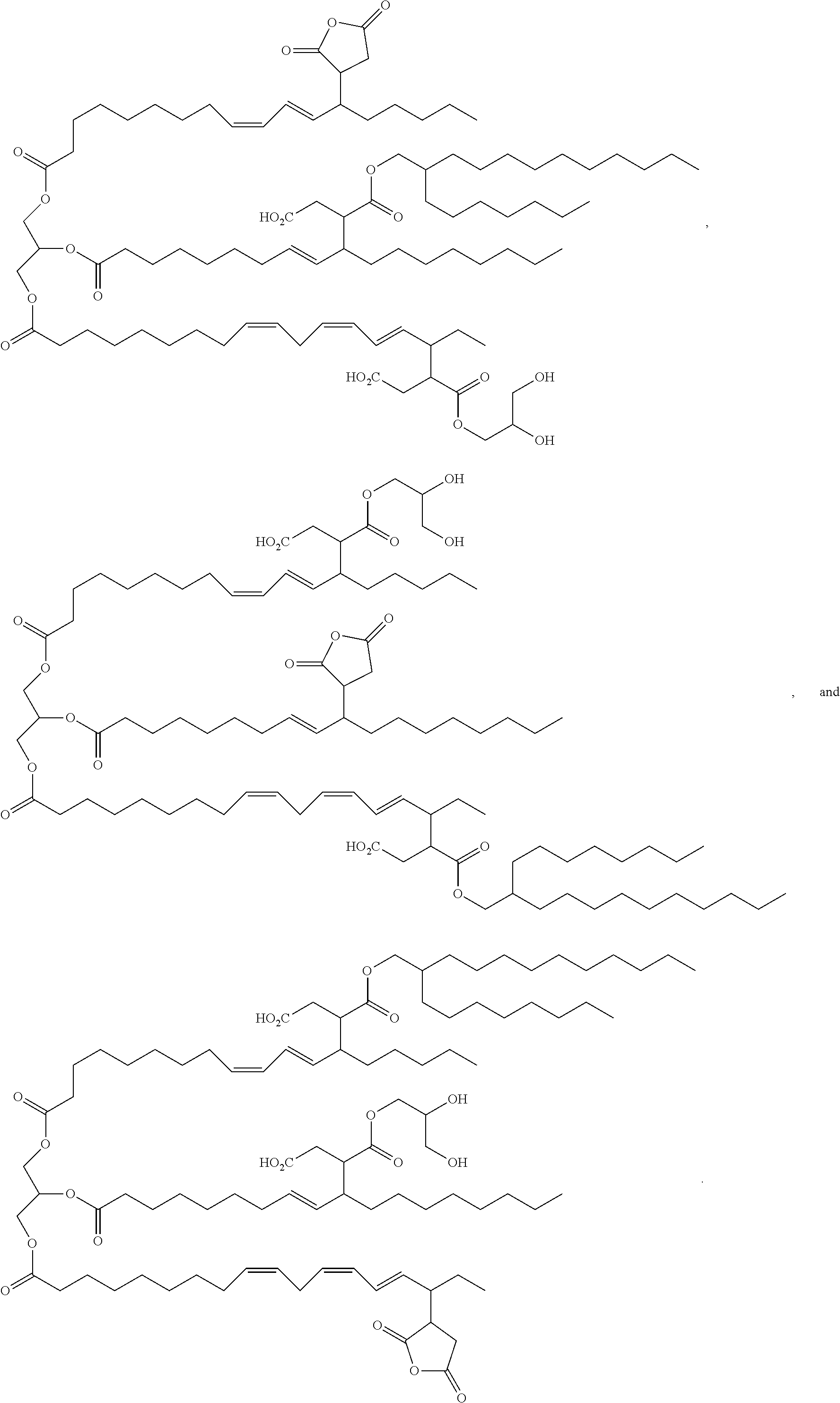

28. A composition comprising:
(A) a reaction product of:
(a) a maleated natural oil, comprising a natural oil with maleated functionality; and
(b) a functionalized or unfunctionalized moiety selected from the group consisting of hydrophobic moieties, hydrophilic moieties, and combinations thereof,
with the proviso that the hydrophilic moiety is not a glycerol moiety
(B) (a) a functional system active ingredient.

29. The composition according to claim 28, wherein the reaction product comprises an unreacted maleated functionality.

30. The composition according to claim 28, wherein the reaction product comprises a maleated functionality functionalized with a hydrophobic moiety, a hydrophilic moiety, and combinations thereof.

31. The composition according to claim 28, wherein the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof.

32. The composition according to claim 28, wherein the hydrophilic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₁ to about C₅ atoms, unsubstituted or substituted alkyl, cycloalkyl, alkenyl, and aryl amines, wherein any of the beforehand mentioned groups may be with or without heteroatoms, containing from about C₁ to about C₅ atoms, unsubstituted or substituted polyols, wherein any of the beforehand mentioned groups may be with or without heteroatoms, containing from about C₂ to about C₃₆ atoms, silanes, and combinations thereof.

33. The composition according to claim 32, wherein the silane is functionalized with an alcohol or an amine, and combinations thereof.

34. The composition according to claim 31, wherein the hydrophobic alcohol is selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof.

35. The composition according to claim 32, wherein the hydrophilic alcohol is selected from the group consisting of methanol, ethanol, propanol, isopropanol, butanol, ethylene glycol, propylene glycol, diethylene glycol, dipropylene glycol, dibutylene glycol, polyethylene glycol, methoxypolyethylene glycol, polypropylene glycol, hexylene glycol, sorbitol, neopentylglycol, eythritol, mannitol, xylitol, threitol, pentaerythritol, beta-cyclodextrin, L-Ribose, 2-deoxy-D-galactose, and mixtures thereof.

36. The composition according to claim 31, wherein the hydrophobic amine is selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof.

37. The composition according to claim 32, wherein the hydrophilic amine is selected from the group consisting of 2-methylpentane-1,5-diamine, diethanol amine, diisopropanolamine, serinol hydrochloride, 2-amino-2-ethyl-1,3-propanediol, N-methyl-D-glucosamine, D-galactosamine hydrochloride, D-glucosamine hydrochloride, D-mannosamine hydrochloride, and mixtures thereof.

38. The composition according to claim 31, wherein the silicon-based compound is a hydrophobic compound selected from the group consisting of aminopropylmethylsiloxane-dimethylsiloxane, N-ethylaminoisobutyl terminated polydimethylsiloxane, poly(1,1-dimethyl silazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

39. The composition according to claim 32, wherein the silane is a hydrophilic compound selected from the group consisting of 3-aminopropylsilanetriol, N-(2-aminoethyl)-3-aminopropylsilanetriol, and mixtures thereof.

40. The composition according to claim 28, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof.

41. The composition according to claim 40, wherein the maleated natural oil is a maleated soybean oil.

42. The composition according to claim 28, wherein the composition is selected from the group consisting of skin care compositions, oral care compositions, hair care compositions, energy compositions, construction compositions, biocidal compositions, preservative compositions, nutraceutical compositions, food compositions, agricultural compositions, coating compositions, cosmetic compositions, homecare compositions, industrial and institutional compositions, textile compositions, laundry compositions, cleaning compositions, and disinfecting compositions.

43. A composition comprising:
(A) a reaction product of
(a) a maleated natural oil, comprising a natural oil with maleated functionality;
(b) a functionalized or unfunctionalized hydrophobic moiety;
(c) a glycerol moiety; and
(B) a functional system active ingredient.

44. The composition according to claim 43, wherein the reaction product comprises an unreacted maleated functionality.

45. The composition according to claim 43, wherein the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof.

46. The composition according to claim 45 wherein the hydrophobic moiety is an alcohol selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof.

47. The composition according to claim 45, wherein the hydrophobic moiety is an amine selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof.

48. The composition according to claim 45, wherein the silicon-based compound is a hydrophobic compound selected from the group consisting of aminopropylmethylsiloxane-dimethylsiloxane, N-ethylaminoisobutyl terminated polydimethylsiloxane, poly(1,1-dimethylsilazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

49. The composition according to claim 43 wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof.

50. The composition according to claim 49, wherein the maleated natural oil is a maleated soybean oil.

51. The composition according to claim 43, wherein the composition is selected from the group consisting of skin care compositions, oral care compositions, hair care compositions, energy compositions, construction compositions, biocidal compositions, preservative compositions, nutraceutical compositions, food compositions, agricultural compositions, coating compositions, cosmetic compositions, homecare compositions, industrial and institutional compositions, textile compositions, laundry compositions, cleaning compositions, and disinfecting compositions.

52. The composition according to claim 43, wherein the compound is selected from the group of structures represented by the structures set out below:

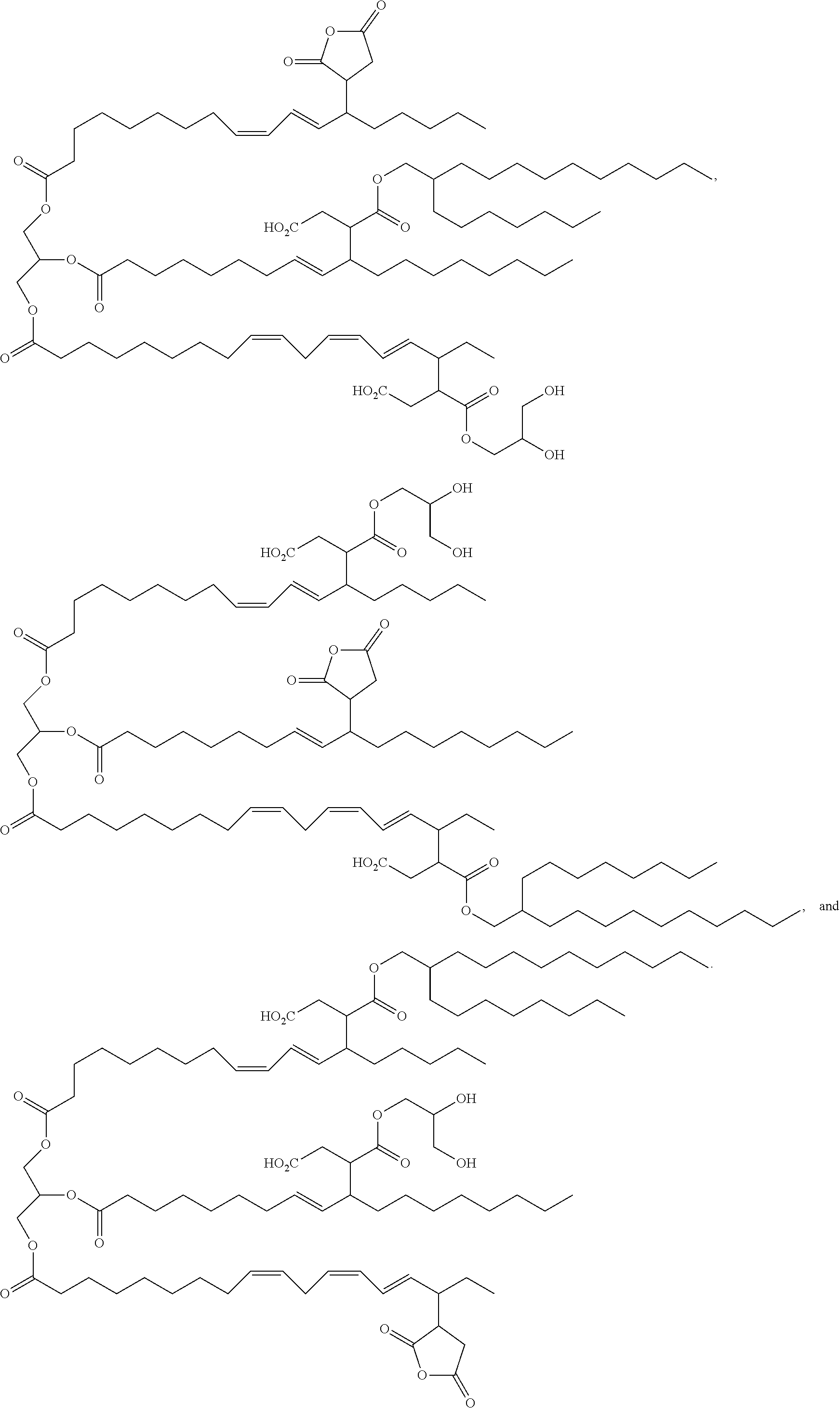

53. A personal care composition comprising:
(A) a reaction product of:
(a) a maleated natural oil, comprising a natural oil with maleated functionality; and
(b) a functionalized or unfunctionalized moiety selected from the group consisting of hydrophobic moieties, hydrophilic moieties, and combinations thereof,
with the proviso that the hydrophilic moiety is not a glycerol moiety; and
(B) (a) a personal care functional system active ingredient.

54. The composition according to claim 53, wherein (A) is present in an amount of from about 0.01% to about 10% and (B) is present in an amount of from about 90% to about 99.99%.

55. The composition according to claim 53, wherein the reaction product comprises an unreacted maleated functionality.

56. The composition according to claim 53, wherein the reaction product comprises a maleated functionality functionalized with a hydrophobic moiety, a hydrophilic moiety, and combinations thereof.

57. The composition according to claim 53, wherein the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof.

58. The composition according to claim 53, wherein the hydrophilic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₁ to about C₅ atoms, unsubstituted or substituted alkyl, cycloalkyl, alkenyl, and aryl amines, wherein any of the beforehand mentioned groups may be with or without heteroatoms, containing from about C₁ to about C₅ atoms, unsubstituted or substituted polyols, wherein any of the beforehand mentioned groups may be with or without heteroatoms, containing from about C₂ to about C₃₆ atoms, silanes, and combinations thereof.

59. The composition according to claim 58, wherein the silane is functionalized with an alcohol or an amine, and combinations thereof.

60. The composition according to claim 57, wherein the hydrophobic alcohol is selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof.

61. The composition according to claim 58, wherein the hydrophilic alcohol is selected from the group consisting of methanol, ethanol, propanol, isopropanol, butanol, ethylene glycol, propylene glycol, diethylene glycol, dipropylene glycol, dibutylene glycol, polyethylene glycol, methoxypolyethylene glycol, polypropylene glycol, hexylene glycol, sorbitol, neopentylglycol, eythritol, mannitol, xylitol, threitol, pentaerythritol, beta-cyclodextrin, L-Ribose, 2-deoxy-D-galactose, and mixtures thereof.

62. The composition according to claim 57, wherein the hydrophobic amine is selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof.

63. The composition according to claim 58, wherein the hydrophilic amine is selected from the group consisting of 2-methylpentane-1,5-diamine, diethanol amine, diisopropanolamine, serinol hydrochloride, 2-amino-2-ethyl-1,3-propanediol, N-methyl-D-glucosamine, D-galactosamine hydrochloride, D-glucosamine hydrochloride, D-mannosamine hydrochloride, and mixtures thereof.

64. The composition according to claim 57, wherein the silicon-based compound is a hydrophobic compound selected from the group consisting of aminopropylmethylsiloxane-dimethylsiloxane, N-ethylaminoisobutyl terminated polydimethylsiloxane, poly(1,1-dimethyl silazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

65. The composition according to claim 58 wherein the silane is a hydrophilic compound selected from the group consisting of 3-aminopropylsilanetriol, N-(2-aminoethyl)-3-aminopropylsilanetriol, and mixtures thereof.

66. The composition according to claim 53, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof.

67. The composition according to claim 66, wherein the maleated natural oil is a maleated soybean oil.

68. The composition according to claim 53, wherein the personal care functional system active ingredient is selected from the group consisting of color agents, hair care agents, skin care agents, and sun care agents.

69. A personal care composition comprising:
(A) a reaction product of
(a) a maleated natural oil, comprising a natural oil with maleated functionality;
(b) a functionalized or unfunctionalized hydrophobic moiety; and
(c) a glycerol moiety; and
(B) a personal care functional system active ingredient.

70. The composition according to claim 69, wherein (A) is present in an amount of from about 0.01% to about 10% and (B) is present in an amount of from about 90% to about 99.99%.

71. The composition according to claim 69, wherein the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof.

72. The composition according to claim 71, wherein the hydrophobic moiety is an alcohol selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof.

73. The composition according to claim 71, wherein the hydrophobic moiety is an amine selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof.

74. The composition according to claim 71, wherein the silicon-based compound is a hydrophobic compound selected from the group consisting of aminopropylmethylsiloxane-dimethylsiloxane, N-ethylaminoisobutyl terminated polydimethylsiloxane, poly(1,1-dimethyl silazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

75. The composition according to claim 69, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof.

76. The composition according to claim 75, wherein the maleated natural oil is a maleated soybean oil.

77. The composition according to claim 69, wherein the personal care functional system active ingredient is selected from the group consisting of color agents, hair care agents, skin care agents, and sun care agents.

78. The composition according to claim 69, wherein the personal care composition is a formulation selected from the group consisting of sprays, lotions, mousses, fluids, serums, solutions, suspensions, perms, emulsions, gels, mists, vesicles, dispersions, pastes, creams, solid sticks, shampoos, balms, wipes, milks, foams, jellies and liquids.

79. A skin care composition comprising:
(A) a reaction product of:
(a) a maleated natural oil, comprising a natural oil with maleated functionality; and
(b) a functionalized or unfunctionalized moiety selected from the group consisting of hydrophobic moieties, hydrophilic moieties, and combinations thereof,
with the proviso that the hydrophilic moiety is not a glycerol moiety; and
(B) (a) a skin care functional system active ingredient.

80. The composition according to claim 79, wherein (A) is present in an amount of from about 0.01% to about 10% and (B) is present in an amount of from about 90% to about 99.99%.

81. The composition according to claim 79, wherein the reaction product comprises an unreacted maleated functionality.

82. The composition according to claim 79, wherein the reaction product comprises a maleated functionality functionalized with a hydrophobic moiety, a hydrophilic moiety, and combinations thereof.

83. The composition according to claim 79, wherein the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof.

84. The composition according to claim 79, wherein the hydrophilic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₁ to about C₅ atoms, unsubstituted or substituted alkyl, cycloalkyl, alkenyl, and aryl amines, wherein any of the beforehand mentioned groups may be with or without heteroatoms, containing from about C₁ to about C₅ atoms, unsubstituted or substituted polyols, wherein any of the beforehand mentioned groups may be with or without heteroatoms, containing from about C₂ to about C₃₆ atoms, silanes, and combinations thereof.

85. The composition according to claim 84, wherein the silane is functionalized with an alcohol or an amine, and combinations thereof.

86. The composition according to claim 83, wherein the hydrophobic alcohol is selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof.

87. The composition according to claim 84, wherein the hydrophilic alcohol is selected from the group consisting of methanol, ethanol, propanol, isopropanol, butanol, ethylene glycol, propylene glycol, diethylene glycol, dipropylene glycol, dibutylene glycol, polyethylene glycol, methoxypolyethylene glycol, polypropylene glycol, hexylene glycol, sorbitol, neopentylglycol, eythritol, mannitol, xylitol, threitol, pentaerythritol, beta-cyclodextrin, L-Ribose, 2-deoxy-D-galactose, and mixtures thereof.

88. The composition according to claim 83, wherein the hydrophobic amine is selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof.

89. The composition according to claim 84, wherein the hydrophilic amine is selected from the group consisting of 2-methylpentane-1,5-diamine, diethanol amine, diisopropanolamine, serinol hydrochloride, 2-amino-2-ethyl-1,3-propanediol, N-methyl-D-glucosamine, D-galactosamine hydrochloride, D-glucosamine hydrochloride, D-mannosamine hydrochloride, and mixtures thereof.

90. The composition according to claim 83, wherein the silicon-based compound is a hydrophobic compound selected from the group consisting of aminopropylmethylsiloxane-dimethylsiloxane, N-ethylaminoisobutyl terminated polydimethylsiloxane, poly(1,1-dimethyl silazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

91. The composition according to claim 84, wherein the silane is a hydrophilic compound selected from the group consisting of 3-aminopropylsilanetriol, N-(2-aminoethyl)-3-aminopropylsilanetriol, and mixtures thereof.

92. The composition according to claim 79, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof.

93. The composition according to claim 92, wherein the maleated natural oil is a maleated soybean oil.

94. The composition according to claim 79, wherein the skin care functional system active ingredient is selected from the group consisting of color agents, hair care agents, skin care agents, and sun care agents.

95. A skin care composition comprising:
(A) a reaction product of
(a) a maleated natural oil, comprising a natural oil with maleated functionality;
(b) a functionalized or unfunctionalized hydrophobic moiety; and
(c) a glycerol moiety; and
(B) a skin care functional system active ingredient.

96. The composition according to claim 95, wherein (A) is present in an amount of from about 0.01% to about 10% and (B) is present in an amount of from about 90% to about 99.99%.

97. The composition according to claim 95, wherein the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof.

98. The composition according to claim 97, wherein the hydrophobic moiety is an alcohol selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof.

99. The composition according to claim 95, wherein the hydrophobic moiety 7s an amine selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof.

100. The composition according to claim 97, wherein the silicon-based compound is a hydrophobic compound selected from the group consisting of aminopropylmethylsiloxane-dimethylsiloxane, N-ethylaminoisobutyl terminated polydimethylsiloxane, poly(1,1-dimethyl silazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

101. The composition according to claim 95, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof.

102. The composition according to claim 101, wherein the maleated natural oil is a maleated soybean oil.

103. The composition according to claim 95, wherein the skin care functional system active ingredient is selected from the group consisting of color agents, hair care agents, skin care agents, and sun care agents.

104. The composition according to claim 95, wherein the skin care functional system active ingredient is a formulation selected from the group consisting of sprays, lotions, mousses, fluids, serums, solutions, suspensions, perms, emulsions, gels, mists, vesicles, dispersions, pastes, creams, solid sticks, shampoos, balms, wipes, milks, foams, jellies and liquids.

105. A composition comprising a reaction product of
(a) a maleated natural oil, comprising a natural oil with maleated functionality;
(b) two functionalized or unfunctionalized hydrophilic moieties.

106. The composition according to claim 105 wherein the reaction product comprises an unreacted maleated functionality.

107. The composition according to claim 105, wherein the hydrophilic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₁ to about C₅ atoms, unsubstituted or substituted alkyl, cycloalkyl, alkenyl, and aryl amines, wherein any of the beforehand mentioned groups may be with or without heteroatoms, containing from about C₁ to about C₅ atoms, unsubstituted or substituted polyols, wherein any of the beforehand mentioned groups may be with or without heteroatoms, containing from about C₂ to about C₃₆ atoms, silanes, and combinations thereof.

108. The composition according to claim 107, wherein the silane is functionalized with an alcohol or an amine, and combinations thereof.

109. The composition according to claim 107, wherein the hydrophilic alcohol is selected from the group consisting of methanol, ethanol, propanol, isopropanol, butanol, ethylene glycol, propylene glycol, diethylene glycol, dipropylene glycol, dibutylene glycol, polyethylene glycol, methoxypolyethylene glycol, polypropylene glycol, hexylene glycol, sorbitol, neopentylglycol, eythritol, mannitol, xylitol, threitol, pentaerythritol, beta-cyclodextrin, L-Ribose, 2-deoxy-D-galactose, and mixtures thereof.

110. The composition according to claim 107, wherein the hydrophilic amine is selected from the group consisting of 2-methylpentane-1,5-diamine, diethanol amine, diisopropanolamine, serinol hydrochloride, 2-amino-2-ethyl-1,3-propanediol, N-methyl-D-glucosamine, D-galactosamine hydrochloride, D-glucosamine hydrochloride, D-mannosamine hydrochloride, and mixtures thereof.

111. The composition according to claim 107, wherein the silane is a hydrophilic compound selected from the group consisting of 3-aminopropylsilanetriol, N-(2-aminoethyl)-3-aminopropylsilanetriol, and mixtures thereof.

112. The composition according to claim 105, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof.

113. The composition according to claim 112, wherein the maleated natural oil is a maleated soybean oil.

114. The composition according to claim 105, wherein the composition is selected from the group consisting of skin care compositions, oral care compositions, hair care compositions, energy compositions, construction compositions, biocidal compositions, preservative compositions, nutraceutical compositions, food compositions, agricultural compositions, coating compositions, cosmetic compositions, homecare compositions, industrial and institutional compositions, textile compositions, laundry compositions, cleaning compositions, and disinfecting compositions.

115. A composition comprising
(A) a reaction product of
(a) a maleated natural oil, comprising a natural oil with maleated functionality;
(b) two functionalized or unfunctionalized hydrophilic moieties; and
(B) a functional system active ingredient.

116. The composition according to claim 115, wherein the functional system active ingredient is a personal care functional system active ingredient.

117. The composition according to claim 115, wherein the functional system active ingredient is a skin care functional system active ingredient.
